(19) United States
(12) Patent Application Publication
(10) Pub. No.: US 2004/0260478 A1
(43) Pub. Date: Dec. 23, 2004

(54) System, process and diagnostic arrangement establishing and monitoring medication doses for patients

(76) Inventor: Lee H. Schwamm, Newton, MA (US)

Correspondence Address: DORSEY & WHITNEY LLP, INTELLECTUAL PROPERTY DEPARTMENT, 250 PARK AVENUE, NEW YORK, NY, 10177, US

(21) Appl. No.: 10/486,171

(22) PCT Filed: Apr. 1, 2002
(86) PCT No.: PCT/US02/10088

Related U.S. Application Data
(60) Provisional application No. 60/310,163, filed on Aug. 3, 2001.

(51) Int. Cl. G06F 17/60; C12Q 1/68; G06F 19/00; G01N 33/48; G01N 33/50

(52) U.S. Cl. 702/20; 705/3; 435/6

(57) ABSTRACT

A diagnostic device, process, system and software arrangement are provided for ascertaining particular information (e.g., medical dosage recommendation and/or therapeutic recommendation) related to a patient. In particular, a diagnostic device can receive previously-stored data associated with the patient from a remote device. Then, a test to ascertain the particular information based on the received previously-stored data can be performed on a biological property (e.g., a biological sample). The particular information may include a level of at least one measurement for the biological property, which may be a compound provided in the sample, with the sample containing a bodily fluid of the patient A web access processing arrangement can also be made available such that access to the respective patient associated with the particular information and a further user (e.g., the patient's physician, nurse, relatives etc.) can be provided so that they can view the received particular information or add information to the system manually.

### CROSS-REFERENCE TO A RELATED APPLICATION

This application claims priority from the Provisional Appln. No. 60/310,163 filed on Aug. 3, 2001, the entire disclosure of which is incorporated herein by reference.

### TECHNICAL FIELD OF THE INVENTION

The present invention relates to an establishment and monitoring of medication dosage levels of patients, and more particularly to a system, process, software and diagnostic arrangement which utilize an interactivity between a client device and a server device to provide data for such establishment and monitoring of the medication dosage levels of patients.

### BACKGROUND

Anticoagulation with a drug warfarin (produced as Coumadin® by E. I. duPont de Nemours and Co., Wilmington, Del.) can be used to prevent initial or recurrent cerebral infarction. This is particularly relevant to strokes caused by emboli, accounting for approximately 60% of all ischemic strokes. Warfarin was proven to be superior to aspirin in preventing strokes in atrial fibrillation (a condition affecting up to 10% of people over age 80). Warfarin's role in preventing strokes by other mechanisms is being investigated in a multicenter NINDS-sponsored study. (See “The Effect of Low-Dose Warfarin on the Risk of Stroke in Patients with Non-Rheumatic Atrial Fibrillation, New England Journal of Medicine, 1990, Vol. 323, pp. 1505-1511; and WARSS, APASS, HAS and Genesis Study Groups, “The feasibility of Collaborative Double-Blind Study using an Anticoagulan”, Cerebrovascular Dis., 1997, Vol. 7, pp. 100-112). Warfarin is also used to prevent thromboembolism from deep venous thrombosis in the lower limbs, an important cause of morbidity and mortality.

Thus, the technical aspects of initiating and managing anticoagulation potentially affect approximately 400,000 patients in the United States of America who suffer an ischemic stroke annually.

Unfortunately, due to the complexity of warfarin metabolism and the wide variability in dose-response in the population, the transition from intravenous heparin to warfarin during a subacute stage of stroke management or venous thrombosis may be complicated by excessive or inadequate anticoagulation. Such excessive anticoagulation can lead to systemic or cerebral hemorrhage, while an inadequate anticoagulation can lead to recurrent ischemic events during this vulnerable period. The transition from intravenous heparin to warfarin has traditionally occurred in an “in-hospital” setting. This is because an inadvertent warfarin overdosing or medication interaction in conditions that are outside the control of the hospital may cause bleeding or prolong the length of a hospital stay, thus contributing to increased in-hospital stroke costs. (See E. M. Hylek et al., “Acetaminophen and Other Risk Factors for Excessive Warfarin Anticoagulation”, JAMA, Mar. 4, 1998, Vol. 279(9), pp. 657-662).

Coumadin initiation “loading” strategies must take various factors into account to be successful and to avoid deleterious effects such as bleeding or recurrent clotting. Prior monitoring has been effected by infrequent venipuncture-based coagulation time measurement techniques. However, such monitoring is a labor-intensive process. “Finger-stick” based methods have been developed, and are currently gaining acceptance. These methods reduce the time between the measurement and the result availability from hours down to minutes, therefore reducing the interval to changes in patient dosing. This has the potential to greatly increase patient compliance.

A number of devices have been utilized to read and provide the results for the finger-sticks. The devices that gained particular notoriety are portable diagnostic devices which can be hand-carried by the user (e.g., a physician, physician's assistant, nurse, pharmacist, etc.). Such devices have been described in U.S. Pat. Nos. 5,108,564, 5,366,609 and 5,438,271.

In particular, U.S. Pat. No. 5,108,564 describes a method and apparatus for an amperometric determination of an analyte of biologically important compounds from body fluids (e.g., glucose, cholesterol, etc.) using a disposable electroanalytical cell. U.S. Pat. No. 5,438,271 describes a diagnostic meter which receives a biomedical disposable sample strip or a check strip, with the sample strip including electrically isolated excitation and sense electrodes. The diagnostic meter includes contacts that are positioned to be electrically connected by the sense electrode when the sample strip is inserted into the diagnostic meter. This meter also includes a processor which is coupled to one of the contacts to be able to determine the presence of a reference potential at another one of the contacts when an inserted sense electrode connects the contacts. The processor of this prior art device determines the characteristics of the biosample provided on the sample strip by passing a current through the sample, and obtaining certain impedance readings therefor.

The results provided by the above-described diagnostic devices and those of the manual tests of the biological samples of the patients are then evaluated to establish a maintenance dosage for each such patient. A number of algorithms designed for calculating the induction and maintenance doses of warfarin have been utilized. For example, one particular algorithm uses a simple method to achieve the target international normalized ratio (“INR”), and predicts the maintenance warfarin requirement by using the value measured on the fourth treatment day. (See A. Fennerty et al., “Flexible Induction Dose Regimen for Warfarin and Prediction of Maintenance Dose Regimen”, British Medical Journal, 1984, Vol. 28(6426), pp. 1268-1270.) The above-referenced algorithm and other similar algorithms are capable of predicting the maintenance dose to within 1 mg/d in only 50-70% of the cases, and can overdose elderly patients or those patients with particular severe medical conditions. Revised regimens using lower doses in elderly in-patients may increase the mean time to therapeutic INR slightly but reduce the frequency of complications due to excessive anticoagulation, as described in G. W. Roberts et al., “Comparison of an Age Adjusted Warfarin Loading Protocol with Empherical Dosing and Fennerty's Protocol”, Australian and New Zealand Journal of Medicine, 1999, Vol. 29(5), pp. 731-736, and J. Gedge et al., “A Comparison of a Low-Dose Warfarin Induction Regimen with the Modified Fennerty Regimen in Elderly Inpatients”, Aging, 2000, Vol. 29(1), pp. 31-34.

Several studies have demonstrated the value of algorithms for providing the maintenance dosages using computer systems. However, the progress has not been significant in the area of predictions of daily maintenance doses from the early response to loading doses. One of the drawbacks of the conventional systems is their inability to adequately allow for a transition from the induction (or loading phase) to the maintenance phase of patients. Prior work has been somewhat cumbersome or inaccurate. However, a more sophisticated model for predicting the maintenance warfarin doses derived from the true area under the dose response curve has been previously used. (See Donald B. Williams et al., “A Simple Technique for Predicting Daily Maintenance Dose of Warfarin”, American Journal of Surgery, 1979, Vol. 137(4), pp. 572-576.) The “area under the curve” analysis has been proposed to incorporate a patient's response to the loading procedure as a measure of his or her metabolism, and is also used to establish a daily maintenance dose. However, this method has not previously been practical to utilize due to the complex mathematical calculations involved therewith.

Furthermore, the prior art diagnostic arrangements and devices have been inefficient in their communication with central computers to transmit and receive information there between. One exemplary device is described in U.S. Pat. No. 6,304,797, which provides an automatic medication dispenser with a remote patient monitoring system. The system uses biosensors to collect data from a patient. Thereafter, the collected data is stored in an SQL database provided on the central computer (i.e., a personal computer). After the data is stored, the central computer is disconnected from the biosensors, and the data is evaluated thereby to determine patient's condition. However, the biosensors described in this publication do not receive any data from the central computer, nor do they have any processing capabilities. Thus, the system of U.S. Pat. No. 6,304,797 only allows for the collection of data from the biosensors, but does not allow the biosensors to receive any data provided by the central computer. Also, neither such prior art diagnostic/biosensing devices nor conventional central computers are capable of efficiently enabling a transition from the induction to maintenance phases of patients.

### SUMMARY

One of the objects of the present invention is to overcome the deficiencies of the prior art. This can be accomplished with the diagnostic device system, process and software arrangement of the present invention for providing certain information (e.g., medical dosage recommendation, therapeutic recommendation, etc.) which relates to a particular patient.

In particular, the diagnostic device or another processing device can receive previously-stored data associated with the patient, possibly from a remote device. Then, a test to ascertain the particular information based on the received previously-stored data can be performed. The ascertained particular information may include a level or measurement of a biological property of the patient. This level or measurement may correspond to at least one compound provided in a sample which may contain a bodily fluid of the patient.

In one exemplary embodiment of the present invention, the diagnostic device or another processing device can transmit the particular information to the remote device via a communication network. An electronic request may also be transmitted to the remote device to provide the previously-stored data via the communication network. In this manner, the remote device is capable of transmitting the previously-stored data from a storage arrangement to the diagnostic device, with the remote device being a central processing arrangement, and the storage arrangement being a database connected to the central processing device. The storage arrangement may also be a storage device connected to the diagnostic device. In one exemplary variation of the present invention, the diagnostic device may obtain the previously-stored data from the storage arrangement which is directly connected to the diagnostic device.

In another exemplary embodiment of the present invention, the processing arrangement can be connected to an input device, and if the patient does not have previously-stored data associated therewith, the diagnostic device may request new data for the patient from the input device. Then, the diagnostic device can receive manually- and/or electronically-provided data for the new patient from the input device. Also, the diagnostic device may include a storage arrangement which stores an executable code thereon, as well as a processing arrangement which retrieves the executable code for generating the particular information.

In yet another exemplary embodiment of the present invention, the processing arrangement of the diagnostic device can execute the executable code to perform a loading dosage procedure for the sample. This loading dosage procedure preferably generates a recommended dosage of medication for the patient based on the previously-stored data and a result of the test, which can be modified by a care provider. The loading dosage procedure may provide the recommended loading dosage of the medication for the patient to a plurality of remote devices, which can be a physician's processing device, a pharmacy's processing device, etc. With this exemplary embodiment, a physician is empowered to change the recommended loading dosage of the medication for the patient using the physician's processing device and/or the diagnostic device.

In still another embodiment of the present invention, the indication can be an international normalized ratio (“INR”) for the sample of the patient. The generated INR and the corresponding recommended dosage for the patient can be transmitted to the remote device to be stored on a remote storage device as a portion of the previously-stored data of the patient. The generated INR and the corresponding recommended dosage for the patient may be stored on the storage arrangement as a portion of the previously-stored data of the patient.

According to yet another embodiment of the present invention, if it is determined that the generated INR of the patient is below a lowest value in a predetermined threshold range, a periodic execution of the loading dosage procedure for the patient can be continued. If it is determined that the INR for the sample of the patient is above a highest value of the predetermined threshold range, an alert may be generated to indicate a value of the INR. Also, if it is determined that the INR for the sample of the patient is in the predetermined threshold range, the recommended dosage of the medication corresponding to the INR for the sample of the patient can be recorded to be utilized by a maintenance dosage procedure.

In yet another embodiment of the present invention, wherein the maintenance dosage procedure may generate a maintenance INR for the sample of the patient based on the previously-stored data and the recommended dosage generated by the loading dosage procedure, with the maintenance INR corresponding to a maintenance dosage. The maintenance dosage of the medication for the patient is capable of being provided to a plurality of remote devices, and capable of being modified by a care provider. A user can also modify the maintenance dosage corresponding to the maintenance INR for the patient. The maintenance dosage procedure can generate the maintenance dosage of the medication for the patient based on the previously-stored data and the INR for the patient generated by the loading dosage procedure. The maintenance dosage of the medication for the patient is capable of being provided to a plurality of remote devices. Also, the maintenance dosage procedure may be initiated after the INR for the patient generated by the loading dosage procedure reaches a value within the predetermined threshold.

In still another embodiment of the present invention, the maintenance INR and the corresponding maintenance dosage for the patient can be transmitted to the remote device to be stored on a remote storage device to be at least a portion of the previously-stored data of the patient. In addition, the maintenance INR for the patient may be stored on the storage arrangement to be a portion of the previously-stored data of the patient. Furthermore, if it is determined that the maintenance INR for the patient is outside a predefined range, the maintenance dosage may be modified and the maintenance dosage procedure re-executed to determine if the maintenance INR corresponding to the modified dosage is within the predefined range. A user is capable of modifying the maintenance INR for the patient.

According to still another embodiment of the present invention, the diagnostic device may be a biosensing portable device (e.g., may include a personal digital assistant), and can be connected to an insertion arrangement which is adapted to receive the sample containing the bodily fluid of the patient. When the sample is received in the insertion arrangement, the diagnostic device may provide data corresponding to a composition and amounts of certain elements of the sample.

Also, the diagnostic device and/or the remote device may transmit the particular information to a web processing arrangement to be displayed on a web page associated with the patient, with the web page being accessible by the patient via a secure connection. In this exemplary embodiment, access to the respective patient associated with the particular information and a further user (e.g., the patient's physician, nurse, relatives etc.) can be provided so that they can view the received particular information. It is possible to provide a personal web page for each of the patients so that the particular information for each respective patient is displayed to the respective patient, and not to other patients. The authorized users may be a physician of the respective patient, a pharmacy of the respective patient, a nurse of the respective patient, a relative of the respective patient, etc.

Unless otherwise defined, all technical and scientific terms used herein have the same, or substantially similar, meaning as commonly understood by one of ordinary skill in the art to which the present invention belongs. Although methods and materials similar or equivalent to those described herein can be used in the practice or testing of the present invention, exemplary processes and materials are described below. In addition, the systems, devices, processes and examples are illustrative only and are in no way limiting. All cited references are incorporated herein by reference.

For a better understanding of the present invention, together with other and further objects, reference is made to the following description, taken in conjunction with the accompanying drawings, and its scope will be pointed out in the appended claims.

### BRIEF DESCRIPTION OF THE DRAWINGS

For a more complete understanding of the present invention and its advantages, reference is now made to the following description, taken in conjunction with the accompanying drawings, in which:

FIG. 1 is a generalized block diagram illustrating a first exemplary embodiment of a portion of a system according to the present invention in which a plurality of diagnostic/biosensing devices communicate with a central processing arrangement via one communication device;

FIG. 2 is a generalized block diagram illustrating a second exemplary embodiment of a portion of the system according to the present invention in which each of the diagnostic/biosensing devices communicates with the central processing arrangement via a respective communication device;

FIG. 3 is a schematic diagram of an exemplary embodiment of the diagnostic/biosensing device according to the present invention;

FIG. 4 is a generalized block diagram illustrating an exemplary embodiment of another portion of the system according to the present invention in which the central processing arrangement communicates with the health care provider and web site equipment;

FIG. 5 is a flow diagram of an exemplary embodiment of a loading procedure for patients according to the present invention;

FIG. 6 is a sample graph of an international normalized ratio vs. cumulative warfarin dose to provide an “area under the curve” which can be used for determining an appropriate maintenance dose for the patient;

FIG. 7A is a flow diagram of an exemplary embodiment of an initial maintenance dosage procedure for the patients according to the present invention which uses the area under the curve shown in FIG. 6, and the results obtained from the loading procedure of FIG. 5;

FIG. 7B is a flow diagram of an exemplary embodiment of a continuation maintenance dosage procedure for the patients according to the present invention which uses the results obtained from the initial maintenance dosage procedure of FIG. 7A;

FIG. 8A is a detailed flow diagram of an exemplary technique to obtain a loading medication dosage of the procedure of FIG. 5 and/or a recommended maintenance dosage of the procedure of FIG. 7B;

FIG. 8B is further details that can be implemented by the continuation maintenance dosage procedure of FIG. 7B;

FIG. 9 is a flow diagram of an exemplary embodiment of the process according to the present invention for obtaining an appropriate level of a bodily fluid;

FIG. 10 is a first exemplary display generated by the system of the present invention in which initial data for the patient can be entered or obtained;

FIG. 11 is a second exemplary display generated by the system of the present invention during the loading procedure illustrated in FIG. 5;

FIG. 12 is a third exemplary display generated by the system of the present invention which illustrates the completion of the loading procedure by the patient.

FIG. 13 is a fourth exemplary display generated by the system of the present invention which illustrates the initiation of the maintenance dose procedure of FIG. 7; and

FIG. 14 is an exemplary display of a record which illustrates the history of the loading procedure, and the output of the maintenance loading procedure of FIG. 7.

### DETAILED DESCRIPTION

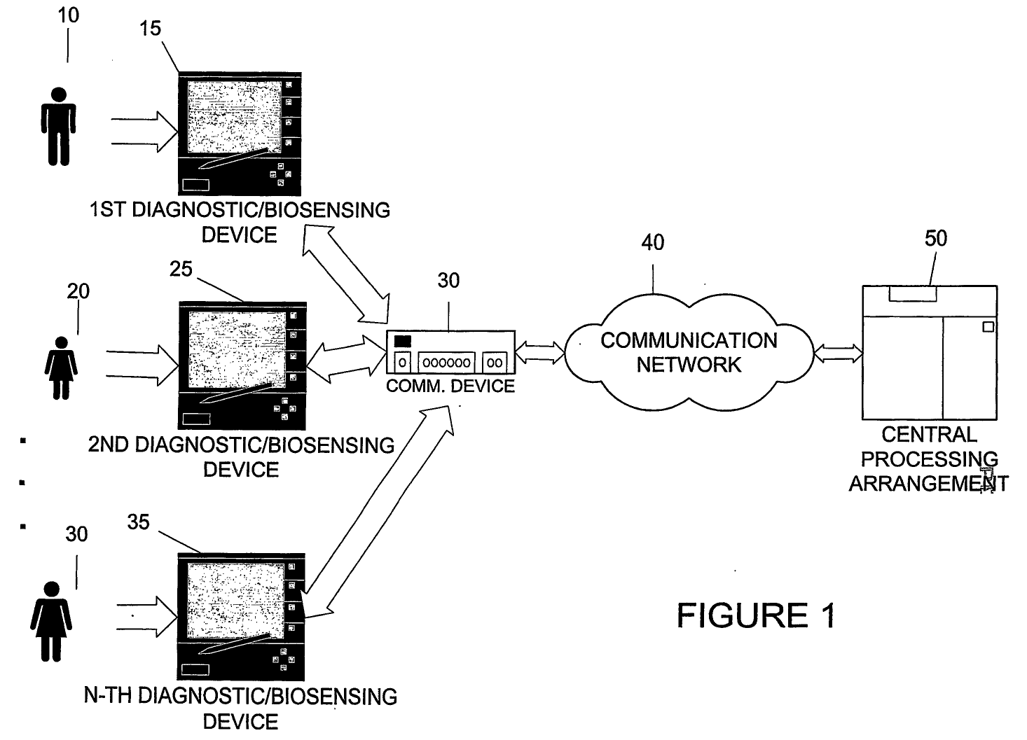

The diagnostic/biosensing device web arrangement, system and process according to the present invention are provided to ease the access of the patient and the health care provider to obtain results of tests of patient's biologically important compounds for his or her body fluids. A first exemplary embodiment of a portion of the system of the present invention is illustrated in FIG. 1, which shows a plurality of diagnostic/biosensing devices 15, 25, 35 each connected to a communication device 30 via a wired connection (e.g., cable connection—analog or digital) or a wireless communication (e.g., analog or digital—TDMA or CDMA). The communication device 30 is connected in the manner described above to a communication network 40 (e.g., the Internet, intranet, local area network, wide area network, etc.), which allows communication between a central processing arrangement 50 and the communication device 30 to take place.

The communication device 30 can be a multiplexer which allows the various signals provided to and from the diagnostic/biosensing devices 15, 25, 35 to be properly relayed to the respective devices, and to be associated with an appropriate diagnostic device which transmits data to and/or receives data from the central processing arrangement 50. The communication device 30 can also be the user's or patient's personal computer which receives the information from and transmits the data to the diagnostic/biosensing devices 15, 25, 35. It is also within the scope of the present invention that the communication device 30 can include a particular number of inputs, this number being equal to or greater than the number of the diagnostic devices. In such manner, it is possible to connect the necessary number of the diagnostic/biosensing devices to the communication device 30. The central processing arrangement 50 can be a general purpose computer (e.g., a Pentium®-based personal computer), a mini-computer, a mainframe, a multiple of each or a combination thereof.

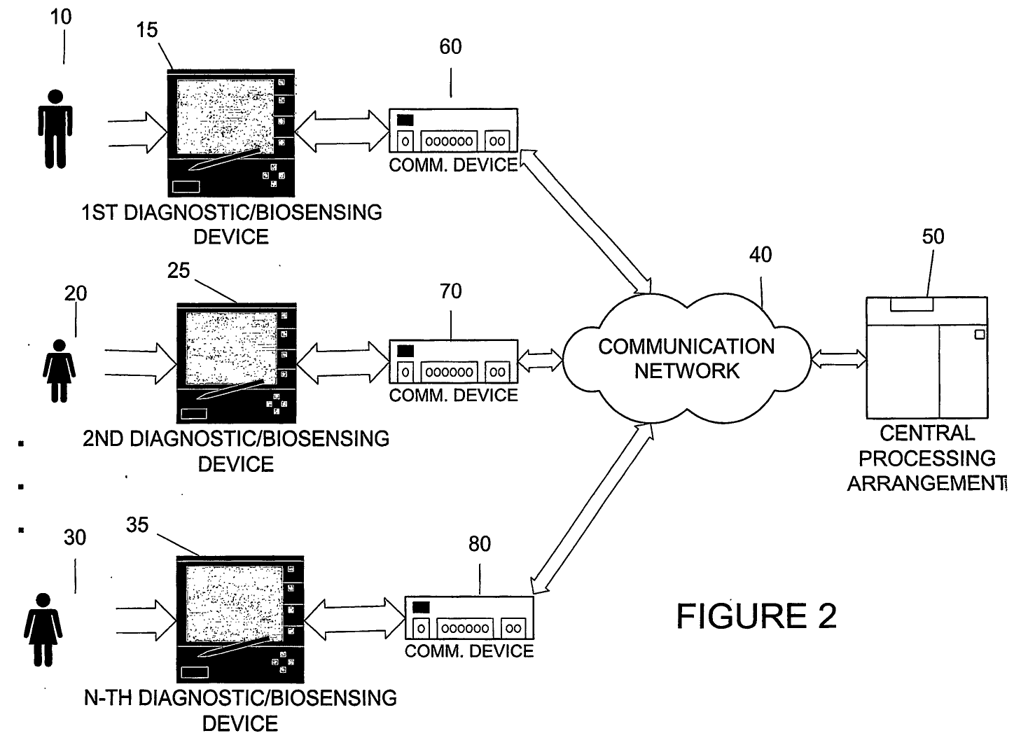

FIG. 2 shows a second exemplary embodiment of the portion of the system according to the present invention. In this second exemplary embodiment, instead of being connected to one communication device 30, the diagnostic devices 15, 25, 35 are each connected to a separate communication device 60, 70, 80, respectively. Thus, each of the communication devices 60, 70, 80 may provide data to and from the respective diagnostic device 15, 25, 35, without the need for each of the communication devices 60, 70, 80 to multiplex or combine the signals coming from and going to the diagnostic/biosensing devices. This arrangement is different from the first exemplary embodiment of FIG. 1 in that while one communication device 30 shown therein was handling the transfer of data to and from the plurality of the diagnostic/biosensing devices 15, 25, 35, each biosensing device being associated with a separate respective communication device. It is also within the scope of the present invention to utilize a variety of connections between the diagnostic devices and the communication devices. For example, two or three devices can be connected to one communication device, and another diagnostic/biosensing device can be connected to yet another separate communication device. It should be understood that other connection arrangements between the diagnostic devices 15, 25, 35 and the communication devices 30, 60, 70, 80 are conceivable.

The diagnostic/biosensing devices 15, 25, 35 can be any sensing devices which obtain the patient's biologically important compounds from his or her body fluids. For example, the first diagnostic/biosensing device 15 can collect this information from a first patient 10, the second diagnostic/biosensing device 25 can collect such data from a second patient 20, and the n-th diagnostic device 35 can collect the biologically-important data from the n-th patient 30. Each of the diagnostic devices 15, 25, 35 can include components of glucometer devices as described in U.S. Pat. Nos. 5,108,564, 5,366,609 and 5,438,271. Such devices can detect the biological compounds of the respective patient's 10, 20, 30 bodily fluid using a “finger-stick” type pads. In particular, the patient's bodily fluid may be provided unto a thin pad, which is then inserted into the pad reading ports of the respective diagnostic device 15, 25, 35 (as also described in U.S. Pat. No. 5,438,271 in greater detail).

It should be understood by those having ordinary skill in the art that the diagnostic/biosensing devices according to the present invention can obtain measurements and readings from sources other than a sample which includes the bodily fluid of the patient. In particular, such exemplary device may use ultrasound or other non- or minimally-invasive methods to extract biological characteristics from a biological property of the patient. While the biological property may include the sample containing the bodily fluid of the patient, this biological property may be a section of the skin sample of the patient, a section of a bone structure of the patient, soft tissue portion of the patient and other biological structures, either within the patient body, or provided outside thereof.

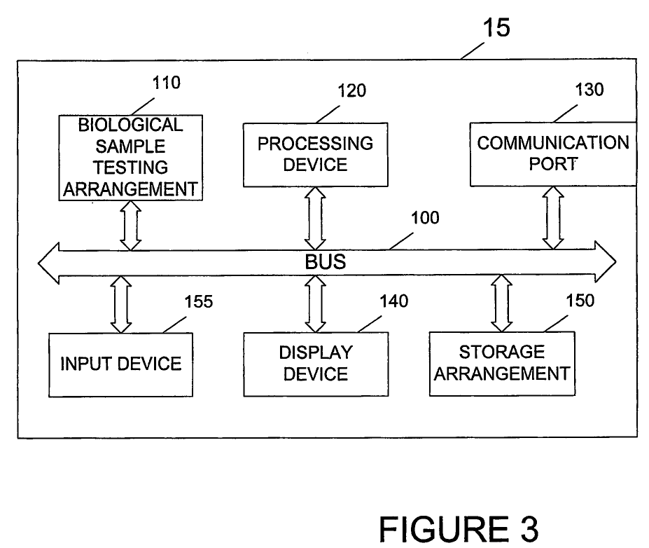

FIG. 3 shows a schematic diagram of an exemplary embodiment of one exemplary device of the diagnostic/biosensing devices 15, 25, 35 according to the present invention. Each of the devices 15, 25, 35 preferably include a biological sample testing arrangement 110 which scans the respective pad inserted in the device and determine data corresponding to the types and amounts of the compounds of the bodily fluid present on the respective pad from the biological sample. This determination can be performed by a processing device 120 which is incorporated into the diagnostic device or connected thereto. The processing device 120 can be a microprocessor, such as a Intel® Pentium®-type processor.

Thereafter, such biological sample data can be forwarded via, e.g., a bus 100 to a display device 140 of the diagnostic device to be displayed thereon, and/or to a storage arrangement 150 of the diagnostic device to be stored thereon. The display device 140 can be a monitor, a liquid crystal display, a printer and the like. The storage arrangement 150 can include read-only memory, random access memory, hard drive, floppy disk drive, compact disk, Read/Write device, etc. The diagnostic/biosensing device 15 can also include an input device 155, such as a keyboard, pen-based arrangement, voice recognition system, etc.) which would allow the operator of the biosensing device 15 to enter information thereon, and/or activate certain features of the diagnostic device thereby sending a signal to the processing device 120 via the bus 100 to perform the requested procedure. It is preferable for the diagnostic/biosensing devices 15, 25, 35 to be portable, i.e., capable of being easily transported from one location to another so that the fluid samples of multiple patients at various locations can be tested.

In operation, upon an occurrence of a predetermined event (e.g., a request by the user, a receipt of new biological sample data on the diagnostic device, a lapse of a predetermined time period, a manual activation, etc.), the processing device 120 directs the biological sample data either directly from the biological sample testing arrangement 110 or from the storage arrangement 150 to a communication port 130 of the diagnostic device via the bus 100. The communication port 130 can be a telephone modem which can be connected to a telephone line, an Ethernet card, a wireless LAN card, etc. The data provided at the communication port 130 can then also be transmitted, via wired or wireless communication, to the communication device 30, 60, 70, 80 (e.g., the patient's or user's personal computer, multiplexer, router, etc.) by wired or wireless communication so as to be forwarded to the central processing arrangement 50 through the communication network 40. Thereafter, the central processing arrangement 50 processes the received information to perform an analysis for determining whether the current dose prescribed to a particular patient (i.e., for whom the analysis was performed) meets certain criteria, as shall be described in further detail below. It is preferable to have the sample diagnostic/biosensing device incorporated in portable personal computers, handheld devices, personal digital assistants, etc. Indeed, such sample diagnostic device can utilize the capabilities, processing power and storage of such portable devices to perform the techniques described above and below.

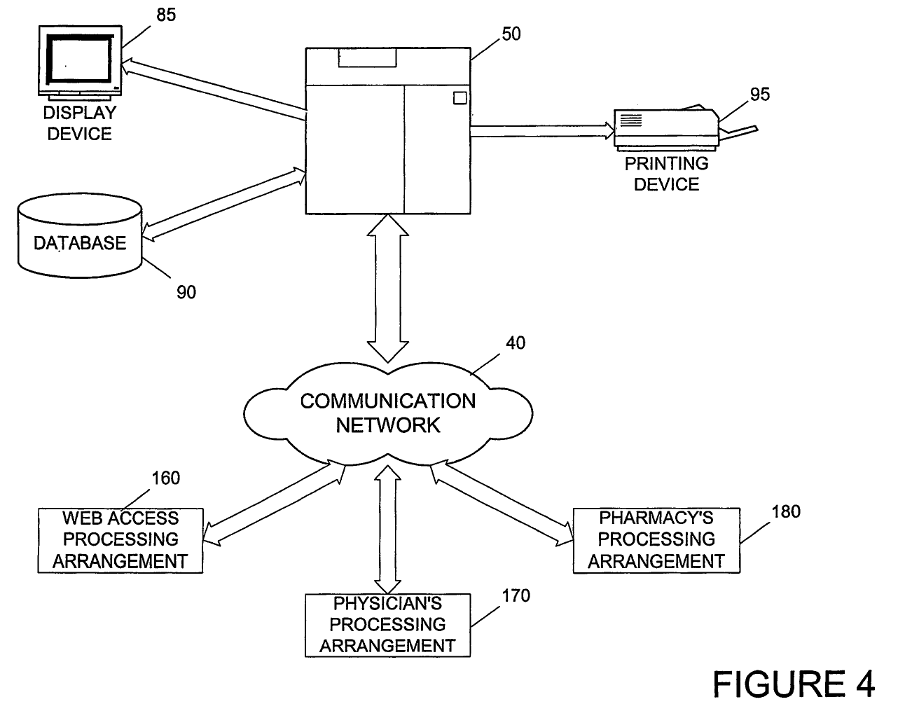

FIG. 4 shows a generalized block diagram illustrating an exemplary embodiment of another portion of the system according to the present invention in which the central processing arrangement 50 performs the above-referenced analysis, display and stores the results, and forwards this information to health care provider devices and web site equipment. In particular, the central processing arrangement 50 is connected to a display device 85, a database 90 and a printing device 95. Thus, upon the central processing device 50's receipt of the data and/or analysis thereof, it can forward the data and/or the results of the analysis to, e.g., the display device 85 to be displayed thereon, the database 90 to be stored thereon and/or the printing device 95 to be printed thereon. In addition, the central processing arrangement 50 preferably forwards the data received from the diagnostic/biosensing devices 15, 25, 35 and/or the information obtained from the analysis made thereby to a web access processing arrangement 160, a physician's processing arrangement 170 and/or a pharmacy's processing arrangement 180. Such forwarding of the data is preferably accomplished via the communication network 40, or another network which may or may not have the features and capabilities of the communication network 40.

If the diagnostic/biosensing device 15, 25, 35 has previously provided the biological sample data for a particular patient to the central processing arrangement 50, any new biological sample data for such patient could be stored on the database 90, e.g., as a list of the sample data for each such patient. In addition, upon the generation of the recommended loading and/or maintenance dosages for the patient, each such generation (i.e., whether initial or continuing) can be stored in this database 90 as a list of the recommended dosages for this patient.

Referring to the web access processing arrangement 160, this arrangement can be at least one web server which may display the data received from the diagnostic/biosensing device, the analysis information, recommended loading dose information, and recommended maintenance dose information for each patient. For example, each of the patients 10, 20, 30 from whom the biological sample was taken can be provided with a secure socket layer (“SSL”) login and communication between the patient's personal computer with the web server. In this manner, each patient may access his or her personal loading and/or maintenance dose information in a secure manner, and possibly obtain further dosage or procedure recommendations. It is within the scope of the present invention to utilize the web access processing arrangement 160 to allow the patient, his/her designee, physicians, laboratories, insurance companies and/or pharmacies to communicate with one another. This can be accomplished by, e.g., allowing the patient's physicians to forward messages and/or instructions to the patient's web page, and the pharmacy to do the same. The processing arrangement 160 of one or more of the diagnostic/biosensing devices 15, 25, 35 also allows the patient or a caregiver to enter the results of the test (e.g., non-finger stick results) to be manually provided thereto, e.g., via the World Wide Web so that sample determinations can also be entered. This may occur while the patient is traveling, on vacation, has no phone line unavailable, located at a conventional blood sampling lab, etc. Such processing arrangement 160 is capable of retrieving a set of rules from the storage arrangement, receive such rules from an external storage device via the communication port 130, and obtain this set of rules from the input device 155 so as to initiate automated communication based on these rules. For example, text paging, fax servers, phone calls, email servers, etc to alert various persons or entities can be implemented based on these rules. As a further example, if the interval between measurements exceeds a specified number of days, or if sample values fell outside a predetermined range, a faxed message can be forwarded to the patient's physician so as to inform him/her of the event.

The physician's processing arrangement 170 can be a personal computer or a mini-computer which would receive the data provided by the respective diagnostic/biosensing devices 15, 25, 35 and/or the analysis data generated by the central processing arrangement 50, store this information for the physician's future reference, print it to be placed with the respective patient's chart, and/or approve/disapprove the recommended doses determined through the analysis of the central processing arrangement 50. When the physician, through the physician's processing arrangement 170, approves or disapproves the recommended loading and/or maintenance dosage for the patient, this information (along with the dosage updated by the physician) is transmitted, e.g., via the communication network 40, to the central processing arrangement 40 so that the physician's approval/disapproval (and possibly the updated dosage) can be stored on the database 90 for future reference. The physician's processing arrangement 170 can also send the modifications to the recommended loading and/or maintenance dosages to the particular patient's web page provided on the web access processing arrangement 160, and to the diagnostic/biosensing device 15, 25, 35.

The pharmacy's processing arrangement 180 can also be a microprocessing device (e.g., a personal computer), a mini-computer or the like, and can be used to receive the recommended loading or maintenance dosages from the central processing arrangement 50. In addition, the pharmacy's processing arrangement 180 can forward the information regarding the availability of the necessary drugs for the recommended loading and/or maintenance dosages to the patient's web page on the web access processing arrangement 160, and then forward the same information to the physician's processing arrangement 170, and to the diagnostic/biosensing device 15, 25, 35. The inter-communication between the web access processing arrangement 160, the physician's processing arrangement 170 and/or the pharmacy's processing arrangement 180 can be achieved through the communication arrangement 40, or via other communication methods, such as wired communication directly between these arrangements, providing the processing arrangements on a LAN or a WAN, wireless communication, etc. This could also generate medication prescription refills at the appropriate dosage and time intervals, and alert the patient, the patient-specified persons, the patient's physician or other health care providers of the renewal.

In one preferred embodiment of the present invention, the diagnostic/biosensing devices 15, 25, 35 are adapted such that the processing devices 120 thereof may transmit requests for information from the central processing arrangement 50. These requests may include the personal information regarding the patient whose biological sample is being tested by the particular diagnostic device (e.g., address, allergies, etc.). For example, the caregiver of the patient may identify himself or herself by providing the patient's identification number (e.g., social security number), using which the central processing arrangement 50 can access the data associated with such patient, and forward the same to the respective diagnostic/biosensing device used for performing the test. In this manner, the test performed for the patient can be associated with the record for the patient at the diagnostic/biosensing device. The patient may also be identified by the using a bar code associated with the patient on the prescription bottle, and then scanning the code with a bar code scanner which would forward the information obtained, and associate the code scanned from the bottle with a particular patient. The bar code scheme can also be used to capture information that is useful for a redundant verification of proper dosing, medication expiration, etc. for the patient.

The information provided by the central processing device 50 to the diagnostic/biosensing devices 15, 25, 35 may also include the current recommended loading/maintenance dosages (and/or the previously recommended loading/maintenance dosages). This information can be displayed to the caregiver and/or the patient on the display device 140 of the respective diagnostic/biosensing device. In this manner, the diagnostic device 15, 25, 35 may not only forward the results of the biological sample test for the patient to the central processing arrangement 50, but also receive the data for the particular patient.

In yet another exemplary embodiment of the present invention, the analysis of the recommended loading and/or maintenance dosages for a particular patient can be performed by the processing device 120 of the diagnostic/biosensing device which is used for performing the test for such patient. For example, such diagnostic/biosensing device 15, 25, 35 may request prior recommended loading and/or maintenance dosages for the particular patient (as described above). Then, upon the receipt of such data, this diagnostic/biosensing device would perform the analysis to determine the new/modified recommended loading and/or maintenance dosage for this patient, and provide the results of the analysis to the patient (via a display on the diagnostic device) and to the central processing arrangement 50 to be stored on the database 90. The details of such analysis shall be described below in further detail.

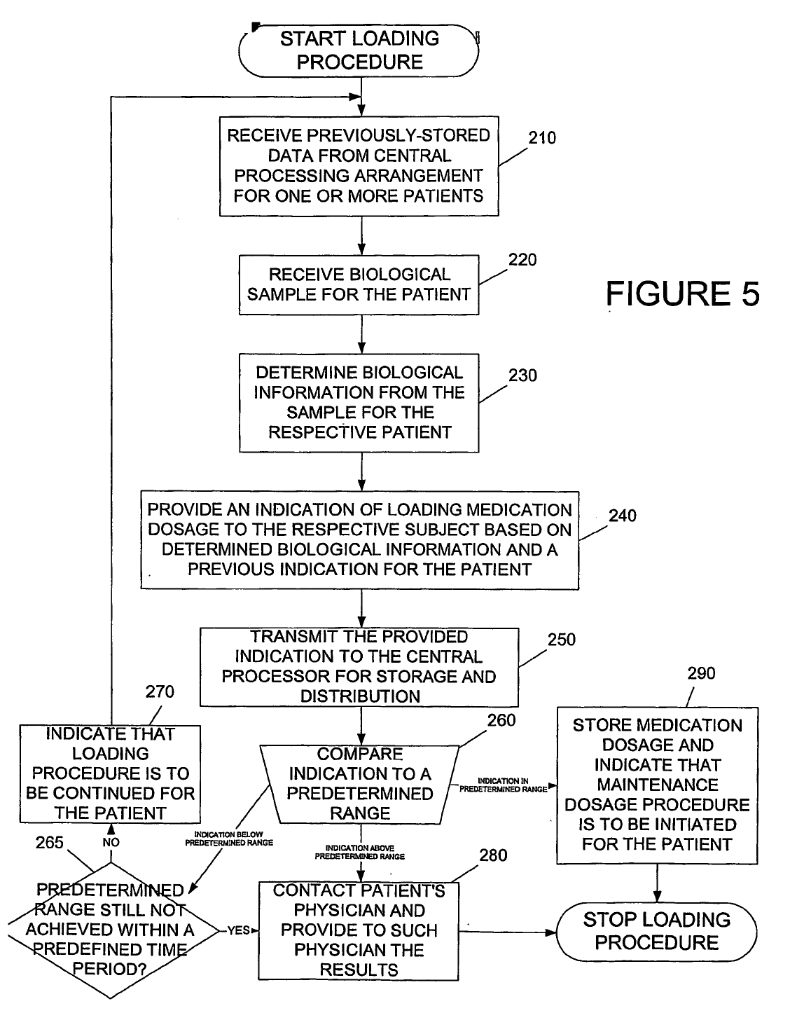

FIG. 5 shows an exemplary embodiment of a loading procedure according to the present invention. When this exemplary procedure is initiated, previously-stored data is obtained from the central processing arrangement 50, a local or remote storage arrangement, etc. (step 210). This previously-stored data may include information corresponding to the patient's previous loading dosage for one or more patients for which the loading procedure is being performed, e.g., patient's demographic information, previous and current medical history, patient's respective initial blood level and international normalized ratio (“INR”), etc. It is also possible to manually enter certain information necessary for the loading procedure associated with such patients, especially for a new patient.

Then, in step 220, a biological sample (associated with the patient being tested) is tested using the diagnostic/biosensing device 15, 25, 35. As indicated above, the biological sample can be provided on the “finger-stick” pad which has the patient's bodily fluids thereon. In step 230, certain biological information is determined from the biological sample for the patient. In one exemplary embodiment of the present invention, the test is performed to determine the patient's the INR to ascertain the patient's coagulation level. Based on the previously-stored data for the patient and the results of the test performed in step 230, an indication can be provided to the tested patient regarding the patient's new INR and new recommended loading medication dosage. This information can be transmitted to the central processing arrangement 50 for storage and distribution in step 250. Then, the central processing arrangement 50 may forward this information to the physician's processing arrangement 170 to obtain an approval or disapproval for the new recommended loading medication dosage. If the physician approves the new recommended loading medication dosage, the central processing arrangement 50 can store this information on the associated database 90, or perform other functions as described in greater detail above.

Thereafter, in step 260, it is determined by the diagnostic device 15, 25, 35 and/or the central processing arrangement 50 whether the new INR is provided below a lowest value of a predetermined threshold range (e.g., the being between the INR level of 2.0 and 3.0), within the predetermined threshold range, or above the highest value of the predetermined threshold range.

In particular, if the INR for the patient is below the lowest value of the predetermined threshold range, then in step 265, it is determined whether the INR has not yet reached the range for longer than a predefined time period (e.g., 3-4 days). If this is the case, the patient's health care provided is contacted, who also receives the results of the tests and loading information conducted to this point (step 280). Thereafter, above-described loading procedure is terminated in favor of conducting another procedure by the health care professional so as to have the patient reach a stable INR for effectively administering the medication, and thereafter possibly a maintenance dosage calculation procedure is initiated (i.e., if the stable INR within the predetermined threshold range is ever reached). If the predefined time period has not has not yet been reached, then a notification or indication is generated stating that the loading procedure is to be continued for the patient in step 270, and the process is forwarded back to step 210 for the re-execution of the loading procedure.

If the INR for the patient is greater than the highest value of the predetermined threshold range, the patient's health care provider is contacted, who also receives the results of the tests and loading information conducted to this point (step 280), and the loading procedure may be terminated in favor of conducting another procedure by the health care professional. In addition or in the alternative, during this period which indicates an unusually high level of response to the medication, a set of rule-based interventions in step 280 also can be triggered (e.g., a recommendation of a medication dose of zero, a change in the frequency of re-testing, etc.) This termination of the loading procedure may be preferable to allow the patient to reach a stable INR for effectively administering the medication, and thereafter possibly initiating a maintenance dosage calculation procedure. Finally, if the INR for the particular patient is within the predetermined threshold range, then in step 290, the medication dosage and the INR for the patient are stored (e.g., in the storage arrangement 160 of at least one of the diagnostic/biosensing devices and/or the database 90) to be used thereafter for the patient.

In a preferred embodiment of the present invention, the central processing arrangement 50 and/or the diagnostic device 15, 25, 35 may require the patient's INR to be steady between 2.0 and 3.0 for authorizing the maintenance dosage procedure to take place in step 270 (using “the area under the curve” analysis). The date of a stable INR level can be defined as the first of 2 consecutive days during which the patient's INR is greater than 2.0, which was not followed by such patient's INR being greater than 3.0 It should be understood that the above-described INR range is only exemplary. Indeed other acceptable ranges (e.g., between 3.0 and 4.0) are conceivable, and are within the scope of the present invention. It is also preferable that the date of the stable INR for the patient is between the third day and the sixth day starting from the first day that the loading procedure took place (e.g., days 3-4, 4-5, or 5-6). Upon reaching the stable INR, the patient can be allowed to undergo the maintenance dosage procedure outside, e.g., a hospital facility. The alternate location where the maintenance dosage procedure can be performed for the patient can be a pharmacy, patient 's residence, doctor's office, etc. This procedure can be administered by a healthcare provider, such as a physician, a physician's assistant, a nurse, a pharmacist, etc. Also, the loading procedure can also be performed in other locations as long as the patient is receiving the appropriate medication, and is not restricted to the in-hospital domain. It is should be understood that the above-described loading procedure can be implemented for establishing a proper glucose level for the patient during an assessment of the patient's pancreatic function.

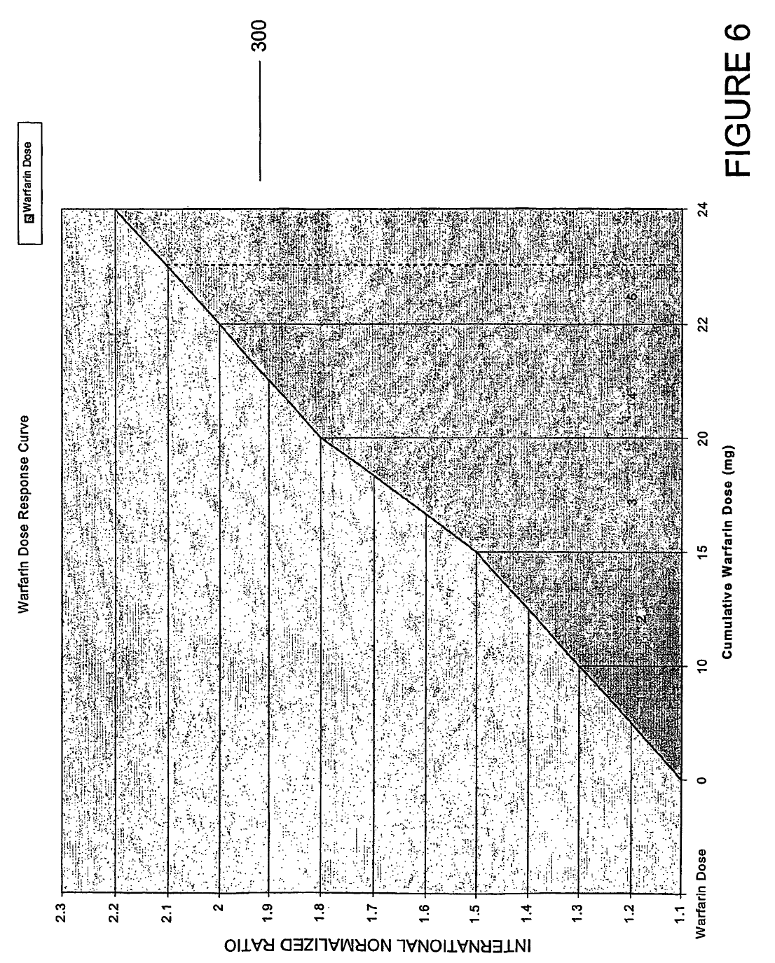

FIG. 6 shows an exemplary graph a warfarin dosage patient-response curve for determining “the area under the curve” for the patients. In this graph, the Y-axis of the graph is a measure of the INR for patients, and the X-axis provides a measure of a cumulative warfarin dosage (in milligrams) which can be used to determine whether the predetermined threshold value of the INR has been reached for the patient. In addition, the dashed line indicates that the INR for the patient exceeded the INR of 2.0 (i.e., at the INR of 2.1) when the patient was administered between 22 and 24 mg of warfarin. It should be understood that this graph is only shown for the purposes of explanation only, and is not intended to limit the scope of the present invention.

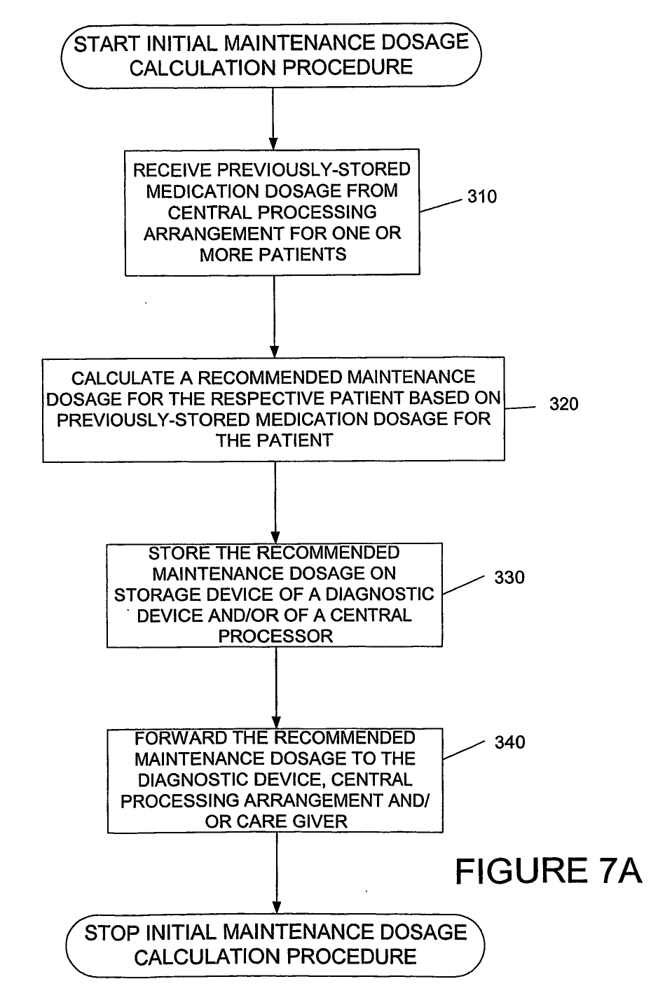

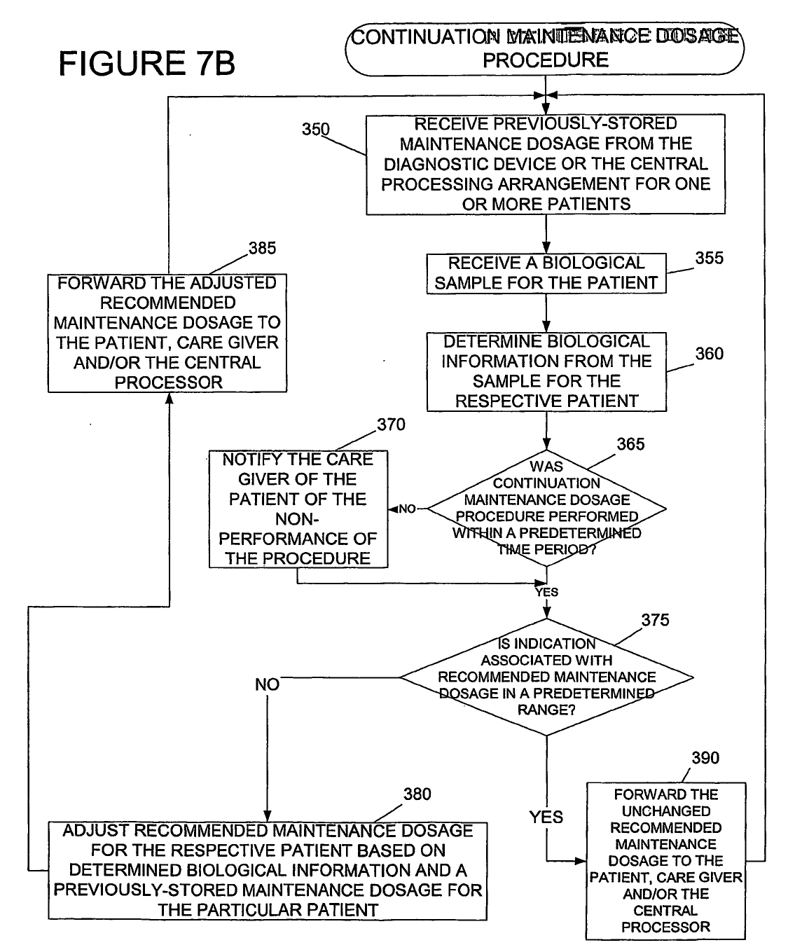

At this point, the execution of the maintenance dosage procedure takes place. In particular, the maintenance dosage procedure consists of an initial maintenance dosage procedure illustrated in FIG. 7A, and a continuation maintenance dosage procedure illustrated in FIG. 7B. Details of each of these maintenance dosage procedures are provided in greater detail herein below.

FIG. 7A shows an exemplary embodiment of the initial maintenance dosage procedure according to the present invention. When this exemplary procedure is initiated, the previously-stored data is obtained from the central processing arrangement 50, the local/remote storage arrangement, etc. (step 310). This previously-stored data may include information corresponding to the patient's previous loading dosage, the patient's previous maintenance dosage for one or more patients for which the maintenance dosage procedure is being performed, patient's demographic information, previous and current medical history, patients' respective initial blood level, and corresponding INR.

Then, in step 320, a recommended maintenance dosage can be calculated by the processing arrangement 160, by one or more of the diagnostic/biosensing devices 15, 25, 35, and/or by the central processing arrangement 50 based on the medication dosages of the patient as recorded during the loading procedure of FIG. 5. The recommended maintenance dosage is stored (e.g., on the storage arrangement 160 of one or more diagnostic/biosensing devices, the database 90, etc.) in step 330. Thereafter, in step 340, the recommended maintenance dosage can be forwarded to the diagnostic/biosensing devices 15, 25, 35, the central processing arrangement 50 and/or other processing arrangements (e.g., the web access processing arrangement 160, the physician's processing arrangement 170, the pharmacy's processing arrangement 180, etc.). At that point, the initial maintenance dosage procedure may be completed.

The continuation maintenance dosage procedure illustrated in FIG. 7B follows the initial maintenance dosage procedure discussed above. In particular, the previously stored maintenance dosage (calculated in the initial maintenance dosage procedure of FIG. 7A) is received for the patient from the storage arrangement 150 of one or more of the diagnostic/biosensing devices 15, 25, 35, the central processing arrangement 50 and/or the database 90 (step 350). In step 355, another biological sample (associated with the patient being tested) is obtained using the diagnostic device 15, 25, 35. In step 360, the biological information is further determined from the biological sample for the patient (similarly to step 220 of FIG. 5).

Thereafter, in step 365, it is determined whether the current continuation maintenance dosage procedure was performed within a predetermined time period from the performance of the previous iteration (if any) of the continuation maintenance dosage procedure. If so, the care provider/giver of the patient can be notified of this fact in step 270, and the procedure is continued (i.e., to step 375). Otherwise, in step 375, it is ascertained if the indication (e.g., INR) associated with the recommended maintenance dosage for the patient within the predetermined threshold range. If that is the case, the recommended maintenance dosage remains unchanged, and it is forwarded to the patient, one or more of the diagnostic/biosensing device(s) 15, 25, 35, the caregiver and/or the central processing arrangement 50, possibly for storage, in step 390, and the processing of the continuation maintenance dosage procedure returns to step 350 for further execution of the continuation maintenance dosage procedure using the unmodified recommended maintenance dosage.

If the indication is not within the predetermined threshold range, the recommended maintenance dosage for the respective patient can be adjusted (i.e., upward or downward) based on determined biological information and the previously-stored maintenance dosage for the particular patient (step 380). Thereafter, the adjusted recommended maintenance dosage is forwarded to the patient, one or more of the diagnostic/biosensing device(s) 15, 25, 35, the patient's care provider/giver (e.g., the physician's processing arrangement 170 to obtain an approval or disapproval from the physician for the new recommended maintenance medication dosage) and/or the central processing arrangement 50 for storage in step 385, and the processing returns to step 350 for further execution of the continuation maintenance dosage procedure using the adjusted recommended maintenance dosage. Indeed, if the physician approves the new recommended maintenance dosage, the central processing arrangement 50 can store this information on the associated database 90, or perform other functions as described in greater detail above and below.

According to the preferred embodiment of the present invention and as indicated above, it is possible for the diagnostic/biosensing device 15, 25, 35 or the physician's processing arrangement 170 to issue an alert indicating that the patient's INR is outside the predetermined threshold range. This alert can be transmitted to the web access processing arrangement 160, the physician's processing arrangement 170, the pharmacy's processing arrangement 180 and the diagnostic device 15, 25, 35 to indicate to the patient and/or healthcare professionals that a situation has arisen in which the patient requires immediate attention (e.g., hospitalization, informing patient's relatives, etc.). Otherwise, after step 385, it will be confirmed that the maintenance dosage procedure will be continued to be performed for this particular patient (at the intervals previously assigned by the central processing arrangement 50, the diagnostic/biosensing device 15, 25, 35 and/or the patient's physician).

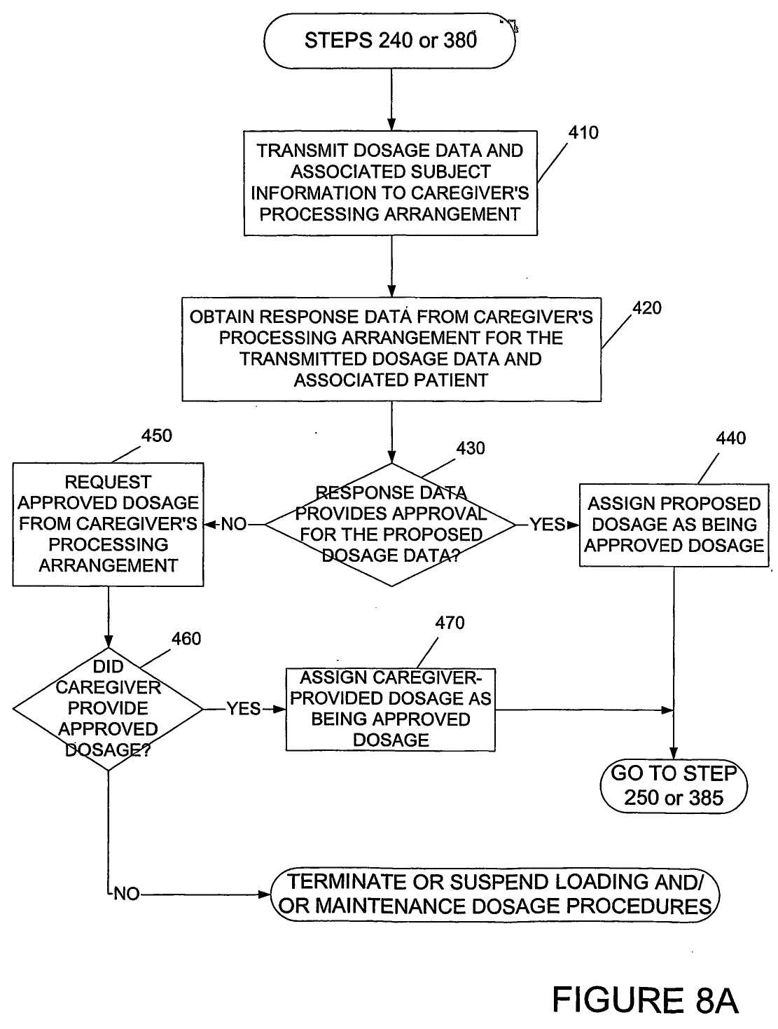

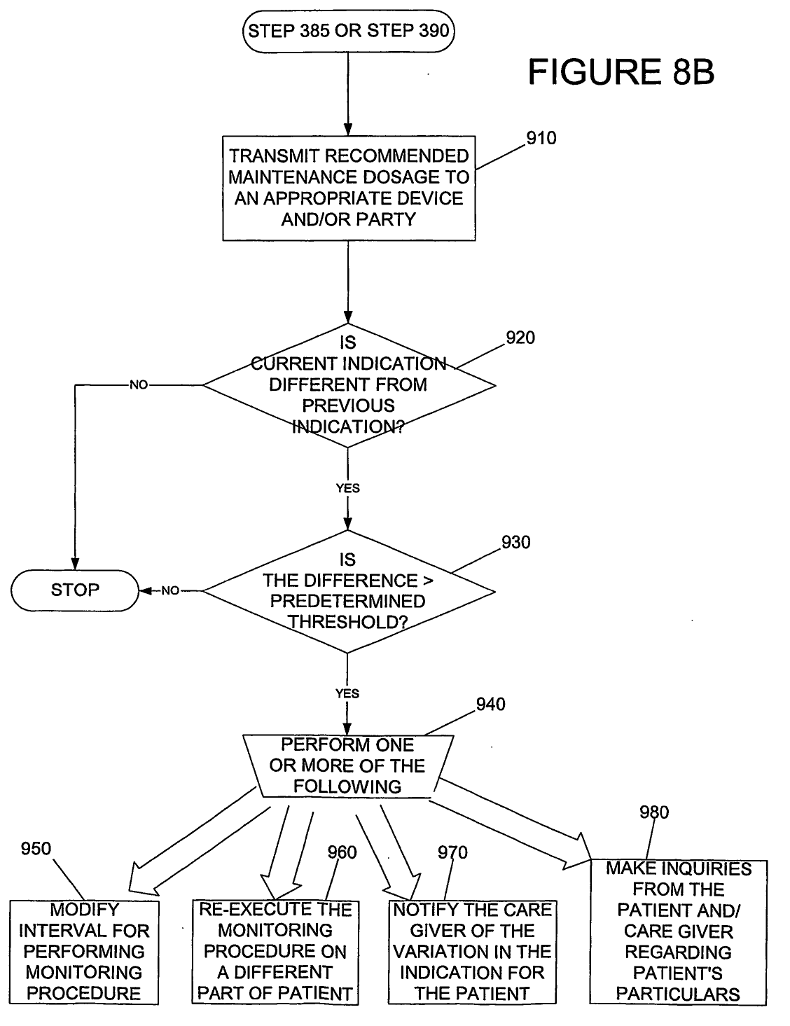

The exemplary details of step 250 (i.e., the step of storage of the medication dosage and indication of the initiation of the initial maintenance dosage procedure of FIG. 5) and step 385 (i.e., the step of providing the recommended maintenance dosage of FIG. 7B) can include the greater details as illustrated in FIG. 8A. In particular, in step 410, the dosage data (e.g., the loading/maintenance dosages) and the associated patient information are transmitted to the caregiver's processing arrangement (e.g., the physician's processing arrangement 170 and the pharmacy's processing arrangement 180). Then, response data is obtained from the caregiver's processing arrangement for the transmitted dosage data and the associated patient (step 420). This response data can be the physician's indication regarding his or her acceptance or rejection of the medication dosages recommended in step 410. In step 430, it is determined whether the response data obtained in step 420 provides approval for the proposed dosage data This can be done by the central processing arrangement 50 and/or the diagnostic/ biosensing device(s) 15, 25, 35. If so, the proposed dosage is marked or assigned as an approved dosage in step 440, and the process returns to the main procedure, i.e., step 250 of the loading procedure or step 310 of the continuation maintenance dosage procedure.

Otherwise, in step 450, the central processing arrangement 50 and/or the diagnostic device(s) 15, 25, 35 request an approved dosage from the caregiver's processing arrangement. If it is determined that the caregiver has provided an approved dosage (step 460), then the dosage provided by the caregiver is marked or assigned as the approved dosage in step 470, and the process returns to the main procedure, i.e., step 250 of the loading procedure or step 350 of the maintenance dosage procedure. Otherwise, the loading/maintenance procedure can be terminated, temporarily suspended, and/or the approved dosage requested from another caregiver or from the same caregiver.

FIG. 8B shows further details that can be implemented by the continuation maintenance dosage procedure described above, especially in steps 385 and 390 thereof. Particularly, in step 910, the recommended maintenance dosage can be transmitted to an appropriate device and/or party (e.g., the patient's computer, the storage arrangement 150 of one or more of the diagnostic/biosensing devices 15, 25, 35, the central processing arrangement 50, the database 90, the web access processing arrangement 160, the physician's processing arrangement 170, the pharmacy's processing arrangement 180, etc.). Then, it is determined in step 920 whether the current indication (e.g., the INR for the particular patient) is different from the previous indication for the patient. If not, this portion of the processing of the continuation maintenance dosage procedure is completed. Otherwise, it is determined if the difference between the current indication and the previous indication is greater than a predetermined threshold value (step 930). If the difference is not greater than this value, this portion of the continuation maintenance dosage procedure is completed. Otherwise, in step 940, the performance of one or more of the particular events can take place. For example, these events are as follows:

the time interval for a periodic performance of the continuation maintenance dosage procedure can be modified (block 950);

the continuation maintenance dosage procedure can be re-executed, e.g., on a different body part of the patient (block 960);

the care provider/giver can receive a notification of the variation of the indication (e.g., the INR) for the patient (block 970); and

inquiries can be made from the patient and/or the care provider/giver regarding the patient's particulars, e.g., has the patient been taking his/her medication, has a new medication recently been started for the patient, has there been a significant change in the patient's diet, etc. (block 980).

As with the loading procedure of FIG. 5, it is should be understood that the above-described initial and continuation maintenance dosage procedures can be implemented for establishing a proper glucose level for the patient during an assessment of the patient's pancreatic function, which is well within the scope of the present invention.

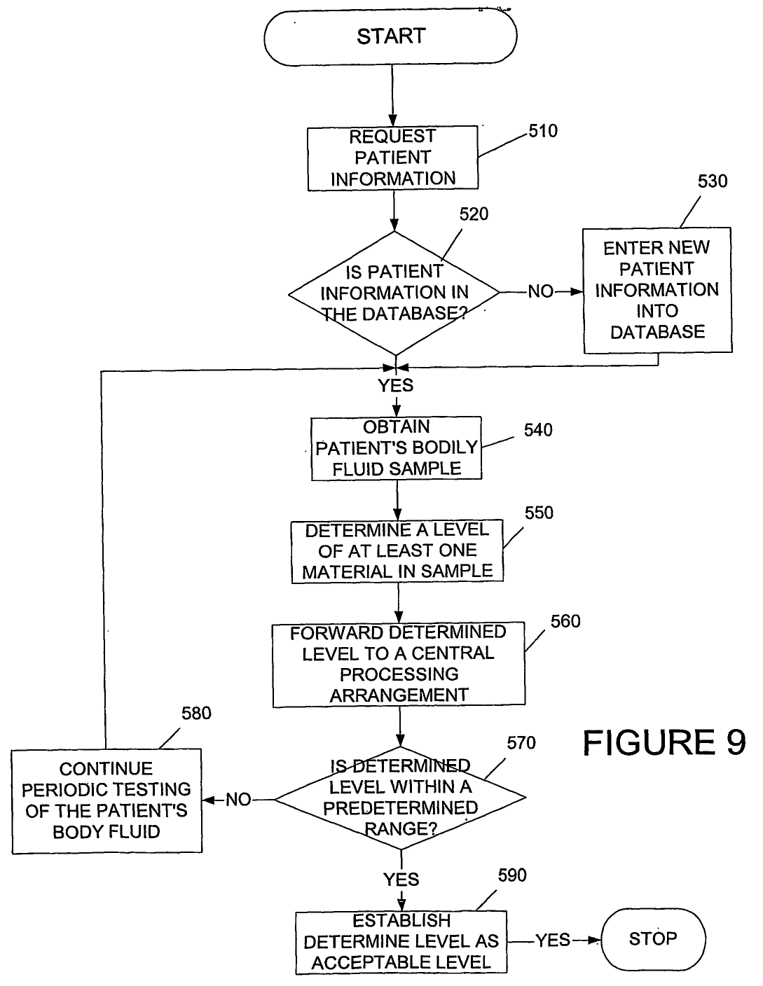

FIG. 9 shows a flow diagram of an exemplary embodiment of the process according to the present invention for obtaining an appropriate level of a bodily fluid of the patient from the sample obtained from such patient. In particular, the patient information can be requested in step 510. This information can be requested by one or more of the diagnostic/biosensing devices 15, 25, 35, the central processing arrangement 50 and/or manually by a caregiver obtaining the sample from the patient. In step 520, it is determined whether the requested patient information is in the database 90 or stored on the storage arrangement 120 of the diagnostic devices 15, 25, 35. If the requested information is not stored on those devices, the requested patient information can be provided either manually or forwarded from external sources, e.g., via the communication network 40 (step 530).

Referring to step 540, the patient's bodily fluid sample can be obtained, e.g., using the “finger-stick” sample. From the patient's bodily fluid, the diagnostic/biosensing device 15, 25, 35 and/or the central processing arrangement 50 can determine the level of at least one material in the sample (e.g., a glucose level) in step 550. Thereafter, the determined level can be forwarded to the central processing arrangement 50, the diagnostic/biosensing devices 15, 25, 35 and/or the caregiver's processing arrangement 170, 180 to be stored on a storage arrangement thereof (or in the database 90). The results can also be forwarded to the web access processing arrangement 160. In step 570, it is then determined whether the determined level has reached a predetermined threshold value or is within a predetermined range (step 570). If so, then the determined level is assigned as the acceptable level by the respective diagnostic/biosensing device 15, 25, 35 and/or the central processing arrangement 50. The caregiver's processing arrangement (and the caregiver in particular) would have the ultimate decision making authorities to establish the acceptability of the determined level.

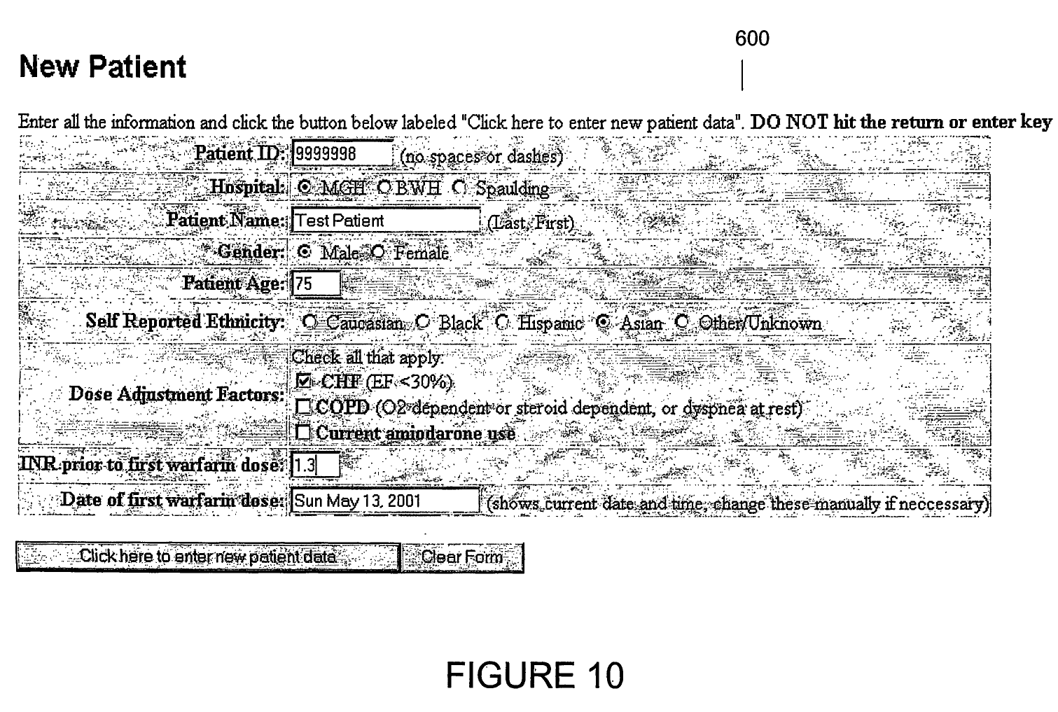

FIGS. 10-14 illustrate the exemplary display screens which assist with the performance and execution of the exemplary embodiment of the process and system according to the present invention. These screens can be generated by the web access processing arrangement 160 to be displayed remotely on the caregiver's, patient's and/or other persons' display devices. In particular, FIG. 10 shows a first exemplary display 600 generated by the system of the present invention in which initial data for the patient can be entered. In this first exemplary display 600, the data can be obtained from the database 90 or the storage arrangement 120 of the diagnostic/biosensing device 15, 25, 35 by, e.g., entering the patient ID or name on the display 600, and then sending a request for information to fill the appropriate locations of the display 600 from the previously stored data for such patient. This information can also be manually or automatically entered, especially for a new patient (see step 530 of FIG. 9). As shown in FIG. 10, the information for the patient may include:

Patient ID

Hospital of the patient

Name of the patient

Gender

Age

Ethnicity

Dose adjustment factors

INR prior to first warfarin dose

Date of first warfarin dose

Of course, it should be understood and within the scope of the present invention that other information for the patient can be present on the display 600 (e.g., patient's address, etc.).

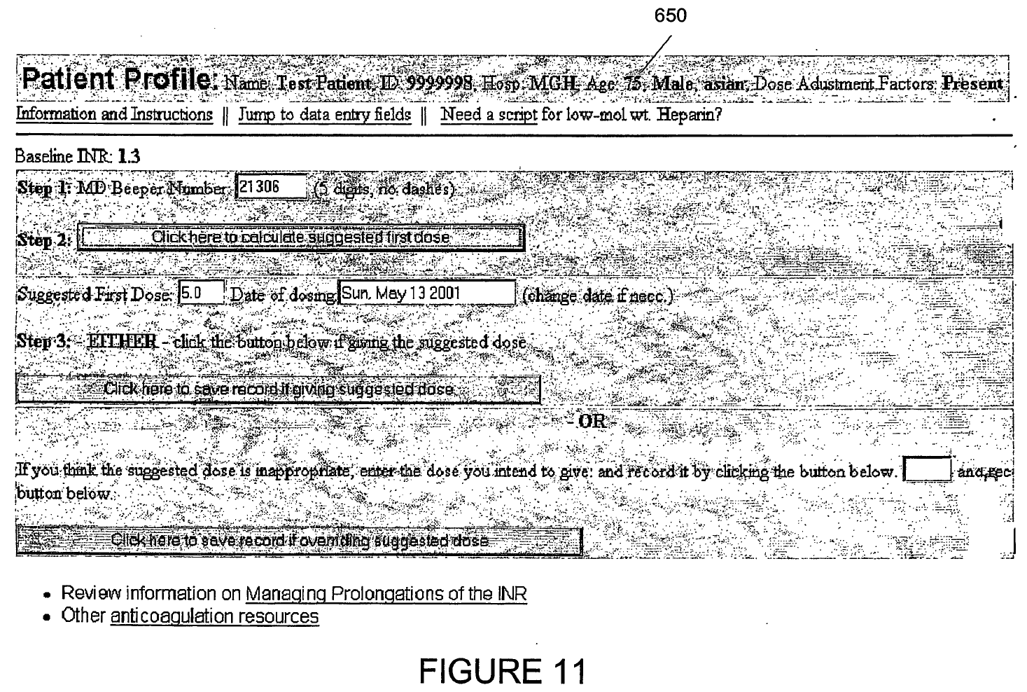

FIG. 11 shows a second exemplary display 650 generated by the system of the present invention during the loading procedure illustrated in FIG. 5. In the display 650, a number of steps can be performed by the user (e.g., the patient's physician, pharmacist, nurse, etc.). For example, the user's identification number can be entered in the first step (e.g., the physician's beeper number). Then, in the second step, the user is given an opportunity to have the suggested first dosage calculated based on the information provided in the first display 600 of FIG. 10. The user is then shown the suggested first dosage and the date of this first dosage, both of which can be modified by this user (e.g., the physician). Thereafter, the dosage (either the calculated first dosage or the dosage modified by the user) can be recorded in the database 90, the storage arrangement 120 of the diagnostic device 15, 25, 35 or another storage device.

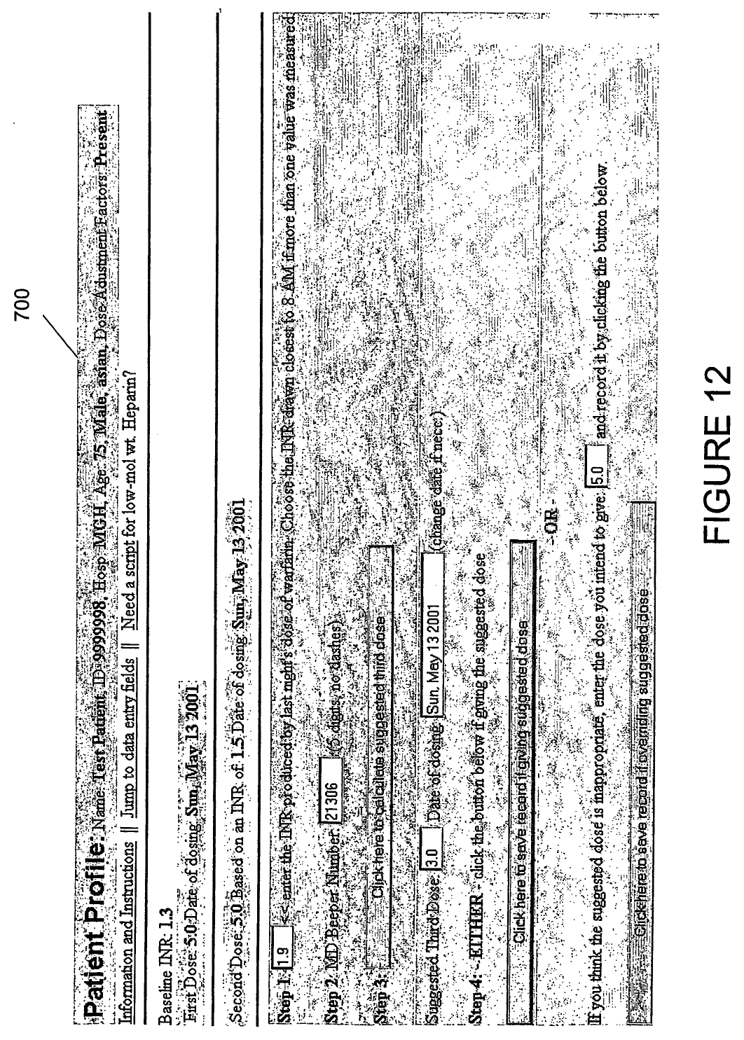

FIG. 12 shows a third exemplary display 700 generated by the system of the present invention which illustrates the completion of the loading procedure by the patient. This display 700 provides the information for the baseline INR, the first dose of warfarin and the date of the first dose. The second dose, the following INR and the date of the second dosing can also be provided on the display 700. It should be understood that third, fourth and subsequent doses and information therefor may also be shown on the display 700.

Also, in this display 700, additional steps are preferably taken by the user. In particular, the user can either obtain another INR produced by, e.g., the previous night's dose of warfarin which may be manually entered or received due to the testing performed by the diagnostic/biosensing devices 15, 25, 35 (the first step). In the second step, the user can enter his or her identification number (e.g., the physician's beeper number) to associate the record with the particular user. Thereafter, in the third step, the user can be allowed to initiate the calculation of the subsequent dose for the patient based on the patient's previous dose and INR information, along with the user's data. Similar to the description provided for the display 650, the subsequent suggested dose and the date of such dose can be provided on the display 700, and the user would have the option and authority to change this information. Then, the subsequent dose and date therefor (either calculated or user-specified) can be recorded in the database 90 of the diagnostic/biosensing device's storage arrangement 120.

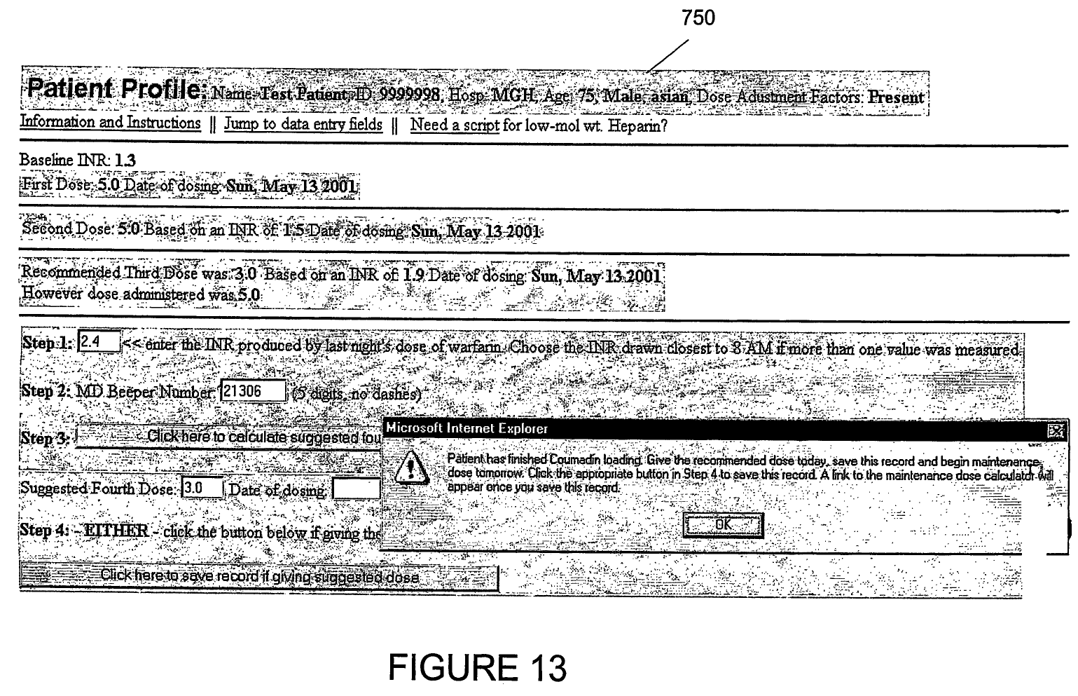

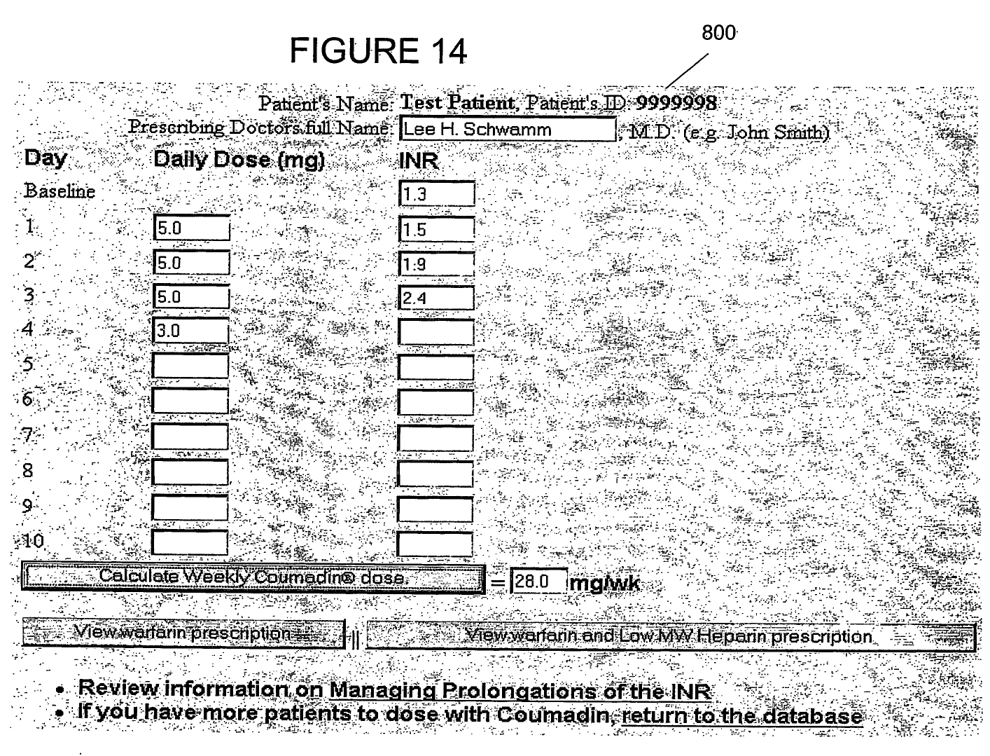

FIG. 13 shows a fourth exemplary display 750 generated by the system and process of the present invention which illustrates the initiation of the maintenance dose procedure of FIG. 7. In particular, the process of the present invention (e.g., as shown in FIG. 5) can be used to establish that the loading procedure has been completed. Thus, the maintenance dosage procedure of FIG. 7 is initiated to calculate the appropriate maintenance dosages of warfarin. A fifth exemplary display 800 generated by the system of the present invention for generating weekly Coumadin doses is shown in FIG. 14.

Upon the calculation and approval of the new loading and/or maintenance dosages or upon the physician's update of the calculated data, the central processing arrangement 50 can forward this information by e-mails to the patient (or provide it to the web access processing arrangement 160 to be viewed by the patient, his or her relative, caregivers, etc.). The caregivers, patient's relatives and friends can communicate with each other and the patient via the web pages of the web access processing arrangement 160 and/or the diagnostic/biosensing devices, portable computing devices, cell phone and/or land phone of the patient. In one preferred exemplary embodiment of the present invention, if the system determines that the international normalized ratio (“INR”) for the patient's coagulation level was substantially out of range, an emergency alert signal can be transmitted by the diagnostic device 15, 25, 35 or the central processing arrangement 50 to the web access processing arrangement 160 and the caregiver's processing arrangement 170, 180 by, e.g., email, pre-recorded phone message, etc.

According to one exemplary embodiment of the present invention, the web access processing arrangement 160 may generate a web page for each patient, such that the respective patient's personal page may be set up to display the patient's current and past dose, INR records, other medicines which could interact with Coumadin, medical information, advances in research, etc. As discussed above, this web page could be transmitted to or allowed to be accessed by other designated persons, such as relatives, health personnel, pharmacies, etc., and can be password protected. In this manner, each patient may obtain his or her information from the web access processing arrangement 160 via the communication network 40. The web page may have the capability to keep track of other medical dosing information corresponding to the patient for retrieval, or can be linked to other electronic medical records. It is also conceivable for the system and process of the present invention to notify the caregiver when the dose change instructions had been read or accessed by the patient, and transmit alerts if recommended clotting measurements had not been performed for such patient or there is a significant change in the recommended dosages.

The system and method of the present invention also makes the passage of information from doctor back to patient an asynchronous event, thus allowing for an uninterrupted flow of information more successfully. Prior art methods and systems require the information passage to take multiple hours, if not days of waiting before the patient's dose is modified. However, a delayed response (i.e., a phase lag) may lead to larger changes in the clotting system parameters. Reducing the time to adjust the dose based on the obtained sample data as provided by the present invention can lead to more efficient patient management, a greater patient compliance and a reduction of the risk to the patient. The system, diagnostic/biosensing device and process of the present invention can also substantially reduce the costs associated with the testing of the patient's sample because the point of care for the dosage management no longer has to be the physician's facility or the laboratory. Indeed, the testing and dosage adjustments can be performed in the facility that is most convenient to the patient (e.g., the patient's home), twenty-four hours a day. Alternatively, the diagnostic/biosensing devices can be located at predetermined local sites such as the pharmacy, church, etc. where they can serve numerous patients more efficiently the utilizing providers which have more experience in diagnostic sampling methods.

The system, diagnostic/biosensing device and process according to the present invention can also be used to monitor the initiation and maintenance of other medications for which the therapy is adjusted based on the level of a substance in a bodily fluid. For example and as described above, the use of insulin or oral hypoglycemic agents in diabetes mellitus can be implemented according to the concepts described herein for the coagulation therapy. In this particular situation, the patients or physicians monitor blood or urine glucose levels frequently, and adjust the doses of medications accordingly. For such patients, the loading dose phase can be used to assess a patient's dose-response to the agent administered (e.g., insulin), and a maintenance dose prediction, dosing interval and testing interval would be established. Different formulations of the medication (e.g., short-acting vs. long-acting) may be selected for different times of day based on one or more rules, and dosage adjustments can be made for each type of the formulation. Values associated the medication levels which fall outside of a predetermined range may trigger an immediate re-measurement thereof, e.g., so as to detect equipment or specimen handling errors. Provided below is an exemplary list of other biological properties which can be measured using the system, process and diagnostic device according to the present invention, the drug class related to this property, and certain examples of the drug(s) for each such class:

| Biological Property to be | Drug class related to | |
|---|---|---|
| measured | compound | Example(s) |
| Potassium | electrolyte supplement oral | K-Dur ® |
| | or parenteral | |
| Sodium | electrolyte supplement oral | NaCl Solution for |
| | or parenteral | injection 0.45% |
| Bicarbonate | electrolyte supplement oral | Neut ® |
| | or parenteral | |
| Calcium | electrolyte supplement oral | Os-Cal ®-500 |
| | or parenteral | |
| Phosphorous | electrolyte supplement oral | Neutra-Phos ®-K |
| | or parenteral | |
| Magnesium | electrolyte supplement oral | Magonate ® |
| | or parenteral | |
| Cholesterol and its | Lipid lowering agent | Pravacho ® |
| subtypes | | |
| Partial thromboplastin | anticoagulant | Hep-Lock ® |
| time | | |
| Ammonia | Ammonium Detoxicant | Cholac ®; |
| Osmolarity | Diuretic, Osmotic | Osmitrol ®; |
| Osmolarity | Antidiuretic Hormone | Pitressin ® |
| | Analog | |
| Blood pressure | Antihypertensive | Inderal ®; |
| | | Inderal ® LA |
| Glucose | Antidiabetic Agent | Di |
| | | a |
| | | Error!Error! |
| | | Unknown switch |
| | | argument.eta ®; |
| Blood urea nitrogen | electrolyte supplement oral | NaCl Solution for |
| | or parenteral | injection 0.45% |
| Thyroid stimulating | Thyroid Product | Levothroid ® |
| hormone | | |
| White blood cells | Colony Stimulating Factor | Neupogen ® |
| White blood cells | Antineoplastic Agent | Cytoxan ®; |
| Red blood cells | Colony Stimulating Factor | Procrit ® |
| CA-125 | Antineoplastic Agent | Taxol ® |
| Phenytoin | Anticonvulsant | Dilantin ® |
| Carbamazepine | Anticonvulsant | Tegretol ® |
| Barbiturate | Anticonvulsant | Nembutal ® |
| Valproic acid | Anticonvulsant | Depakote ® |
| digoxin | Antiarrhythmic Agent | Lanoxin ® |
| gentamicin | Antibiotic | Garamycin ® |
| vancomycin | Antibiotic | Lyphocin ® |

It is to be understood that while the invention has been described in conjunction with the detailed description hereof, the foregoing description is intended to illustrate and not limit the scope of the invention, which is defined by the scope of the appended claims. Other aspects, advantages, and modifications are within the scope of the following claims.

1. A diagnostic device for ascertaining particular information related to a patient, comprising:
a processing arrangement which is capable of:
i. receiving previously-stored data associated with the patient, and
ii. ordering a performance of a test on a biological property of the patient to ascertain the particular information based on the received previously-stored data, the particular information including at least one measurement of the biological property.

2. The diagnostic device according to claim 1, wherein the processing device is capable of transmitting the particular information to a remote device via a communication network.

3. The diagnostic device according to claim 2, wherein the processing arrangement makes an electronic request for the previously-stored data to be provided to the diagnostic device from the remote device via the communication network.

4. The diagnostic device according to claim 3, further comprising:
a communication arrangement connected to the processing arrangement,
wherein the processing arrangement transmits the request for the previously-stored data to the communication arrangement to be forwarded to the remote device via the communication network, and
wherein the communication arrangement receives the previously-stored data from the remote device to be accessed by the processing arrangement.

5. The diagnostic arrangement according to claim 1, wherein the processing arrangement is connected to an input device,
wherein, if the processing arrangement determines that the patient does not have previously-stored data associated therewith, the processing arrangement requests new data for the patient from the input device, and
wherein the processing arrangement receives at least one of manually-entered data or electronically-provided data for the patient from the input device.

6. The diagnostic device according to claim 1, further comprising:
a storage arrangement storing an executable code thereon, wherein the processing arrangement retrieves the executable code to perform the receiving and ordering operations.

7. The diagnostic device according to claim 6, wherein the executable code, when run by the processing device, executes a loading dosage procedure for the sample.

8. The diagnostic device according to claim 7, wherein the loading dosage procedure generates a recommended dosage of medication for the patient when an indication related to the at least one measurement and corresponding to the recommended dosage is within a predetermined threshold range, the recommended dosage being generated based on the previously-stored data and a result of the test.

9. The diagnostic device according to claim 8, wherein the recommended dosage of the medication for the patient is capable of being provided to a plurality of remote devices, and is capable of being modified by a care provider.

10. The diagnostic device according to claim 8, wherein the indication is an international normalized ratio (“INR”) for the sample of the patient.

11. The diagnostic device according to claim 10, wherein the processing arrangement transmits the generated INR and the corresponding recommended dosage for the patient to a remote device to be stored on a remote storage device as a portion of the previously-stored data of the patient.

12. The diagnostic device according to claim 10, wherein the processing arrangement stores the generated INR and the corresponding recommended dosage for the patient on the storage arrangement as a portion of the previously-stored data of the patient.

13. The diagnostic device according to claim 10,
wherein, if the processing arrangement determines that the generated INR of the patient is below a lowest value in a predetermined threshold range, the processing arrangement continues a periodic execution of the loading dosage procedure for the patient,
wherein, if the processing arrangement determines that the INR for the sample of the patient is above a highest value of the predetermined threshold range, an alert is generated to indicate a value of the INR, and
wherein, if the processing arrangement determines that the INR for the sample of the patient is in the predetermined threshold range, the recommended dosage of the medication corresponding to the INR for the sample of the patient is recorded to be utilized by a maintenance dosage procedure.

14. The diagnostic device according to claim 13, wherein the maintenance dosage procedure generates a maintenance INR for the sample of the patient based on the previously-stored data and the recommended dosage generated by the loading dosage procedure, wherein the maintenance INR corresponds to a maintenance dosage.

15. The diagnostic device according to claim 14, wherein the maintenance dosage of the medication for the patient is capable of being provided to a plurality of remote devices, and capable of being modified by a care provider.

16. The diagnostic device according to claim 14, further comprising:
an input device coupled to the processing arrangement, wherein a user is capable of modifying the maintenance dosage corresponding to the maintenance INR for the patient via the input device.

17. The diagnostic device according to claim 14, wherein the maintenance dosage procedure generates the maintenance dosage of the medication for the patient based on the previously-stored data and the INR for the patient generated by the loading dosage procedure.

18. The diagnostic device according to claim 17, wherein the maintenance dosage of the medication for the patient is capable of being provided to a plurality of remote devices.

19. The diagnostic device according to claim 17, wherein the processing arrangement initiates the maintenance dosage procedure after the INR for the patient generated by the loading dosage procedure reaches a value within the predetermined threshold.

20. The diagnostic device according to claim 14, wherein the processing arrangement transmits the maintenance INR and the corresponding maintenance dosage for the patient to a remote device to be stored on a remote storage device to be at least a portion of the previously-stored data of the patient.

21. The diagnostic device according to claim 14, wherein the processing arrangement stores the maintenance INR for the patient on the storage arrangement to be a portion of the previously-stored data of the patient.

22. The diagnostic device according to claim 14, wherein, if the processing arrangement determines that the maintenance INR for the patient is outside a predefined range, the processing arrangement modifies the maintenance dosage and re-executes the maintenance dosage procedure to determine if the maintenance INR corresponding to the modified dosage is within the predefined range.

23. The diagnostic device according to claim 14, further comprising:
an input device coupled to the processing arrangement, wherein a user is capable of modifying the maintenance INR for the patient via the input device.

24. The diagnostic device according to claim 1, wherein the biological property includes a bodily fluid of the patient, further comprising:
an insertion arrangement adapted to receive the bodily fluid, wherein, when the bodily fluid is received in the insertion arrangement, the assertion arrangement makes data, which corresponds to a composition and amounts of certain elements of the sample, available to the processing arrangement.

25. The diagnostic device according to claim 1, wherein the diagnostic device is a biosensing portable device.

26. The diagnostic device according to claim 25, wherein the portable device includes a personal digital assistant.

27. The diagnostic device according to claim 1, wherein the processing arrangement transmits the particular information to a web processing arrangement to be displayed on a web page associated with the patient, the web page being accessible by the patient via a secure connection.

28. A process for determining particular information associated with a patient, comprising the steps of:
a) receiving previously-stored data associated with the patient by a diagnostic device; and
b) with the diagnostic device, performing a test on a biological property of the patient to ascertain the particular information based on the received previously-stored data, the particular information including at least one measurement of the biological property.

29. The process according to claim 28, further comprising the step of:
c) transmitting the particular information to a remote device via a communication network.

30. The process according to claim 29, wherein the transmitting step includes the substep of generating an electronic request by the diagnostic device to the remote device for the previously-stored data to be provided from the remote device via the communication network.

31. The process according to claim 30, wherein step (a) further comprises the substep of forwarding, by the remote device, the previously-stored data from a storage arrangement to the diagnostic device.

32. The process according to claim 31, wherein the remote device is a central processing arrangement, and wherein the storage arrangement is a database connected to the central processing device.

33. The process according to claim 31, wherein the storage arrangement is a storage device connected to the diagnostic device.

34. The process according to claim 30, wherein step (a) further comprises the step of obtaining the previously-stored data by the diagnostic device from a storage arrangement directly connected to the diagnostic device.

35. The process according to claim 28, wherein the processing arrangement is connected to an input device, and further comprising the steps of:
d) if the patient does not have previously-stored data associated therewith, requesting new data for the patient from the input device; and
e) receiving at least one of manually-entered data or electronically-provided data for the new patient from the input device.

36. The process according to claim 28, wherein the diagnostic device includes a storage arrangement which stores an executable code thereon, and further comprising the step of:
f) retrieving the executable code to perform steps (a) and (b).

37. The process according to claim 36, further comprising the step of:
g) running the executable code to executes a loading dosage procedure for the sample.

38. The process according to claim 37, further comprising the step of:
h) generating, with the loading dosage procedure, a recommended dosage of medication for the patient when an indication associated with the at least one measurement and corresponding to the recommended dosage is within a predetermined threshold range, wherein the recommended dosage is generated based on the previously-stored data and a result of a test in step (b).

39. The process according to claim 38, further comprising the step of:
i) providing the recommended dosage of the medication for the patient to a plurality of remote devices, wherein the recommended dosage is capable of being modified by a care provider.

40. The process according to claim 39, wherein the remote devices include at least two of a physician's processing device and a pharmacy's processing device.

41. The process according to claim 40, further comprising the step of:
j) providing a capability for a physician to change the recommended dosage of the medication for the patient using at least one of the physician's processing device and the diagnostic device.

42. The process according to claim 38, wherein the indication is an international normalized ratio (“INR”) for the sample of the patient.

43. The process according to claim 42, further comprising the step of:
k) transmitting the generated INR and the corresponding recommended dosage for the patient to a remote device to be stored on a remote storage device as a portion of the previously-stored data of the patient.

44. The process according to claim 37, further comprising the step of:
l) recording the generated INR and the corresponding recommended dosage for the patient on the storage arrangement as a portion of the previously-stored data of the patient.

45. The process according to claim 37, further comprising the steps of:
m) if the processing arrangement determines that the generated INR of the patient is below a lowest value in a predetermined threshold range, continuing a periodic execution of the loading dosage procedure for the patient;
n) if the processing arrangement determines that the INR for the sample of the patient is above a highest value of the predetermined threshold range, generating an alert to indicate a value of the INR; and
o) if the processing arrangement determines that the INR for the sample of the patient is in the predetermined threshold range, recording the recommended dosage of the medication corresponding to the INR for the sample of the patient to be utilized by a maintenance dosage procedure.

46. The process according to claim 45, further comprising the step of:
p) with the maintenance dosage procedure, generating a maintenance INR for the sample of the patient based on the previously-stored data and the recommended dosage generated by the loading dosage procedure, wherein the maintenance INR corresponds to a maintenance dosage.

47. The process according to claim 46, further comprising the step of:
q) with the maintenance dosage procedure, maintaining the maintenance dosage of the medication for the patient based on the previously-stored data and the INR for the patient generated by the loading dosage procedure.

48. The process according to claim 46, further comprising the step of:
r) providing the maintenance dosage of the medication for the patient to a plurality of remote devices.

49. The process according to claim 46, further comprising the step of:
s) providing an ability for a use to modify the maintenance INR for the patient via an input device.

50. The process according to claim 46, further comprising the step of:
t) providing the maintenance dosage of the medication for the patient to a plurality of remote devices.

51. The process according to claim 50, wherein the remote devices include at least two of a physician's processing device and a pharmacy's processing device.

52. The process according to claim 46, further comprising the step of:
u) initiating the maintenance dosage procedure after the INR for the patient generated by the loading dosage procedure is maintained reaches a value within the predetermined threshold.

53. The process according to claim 46, further comprising the step of:
v) transmitting the maintenance INR for the patient to a remote device to be stored on a remote storage device to be a portion of the previously-stored data of the patient.

54. The process according to claim 46, further comprising the step of:
w) recording the maintenance INR for the patient on a storage arrangement connected to a remote device to be a portion of the previously-stored data of the patient.

55. The process according to claim 46, further comprising the step of:
x) if the maintenance INR for the sample of the patient is outside a predefined range, modifying the maintenance dosage and re-executing the maintenance dosage to determine if the maintenance INR corresponding to the modified dosage is within the predefined range.

56. The process according to claim 46, wherein the diagnostic device is connected to an input device, wherein a user is capable of modifying the maintenance INR for the patient via the input device.

57. The process according to claim 28, wherein the biological property includes a bodily fluid of the patient, and wherein the diagnostic device is connected to an insertion arrangement which is adapted to receive the bodily fluid, and further comprising the step of:
y) when the bodily fluid is received in the insertion arrangement, availing data corresponding to a composition and amounts of certain elements of the sample.

58. The process according to claim 28, wherein the diagnostic device is a portable device.

59. The process according to claim 58, wherein the portable device includes a biosensing personal digital assistant.

60. The process according to claim 28, further comprising the step of:
y) transmitting the particular information to a web processing arrangement to be displayed on a web page associated with the patient, the web page being accessible by the patient via a secure connection.

61. A system for determining particular information associated with a patient, comprising:
a diagnostic device receiving the previously-stored data associated with the patient, the diagnostic device performing a test on a biological property of the patient to ascertain the particular information based on the received previously-stored data, the particular information including at least one measurement of the biological property.

62. The system according to claim 61, wherein the diagnostic device transmits the particular information to a remote device via a communication network.

63. The system according to claim 62, wherein the diagnostic device transmits an electronic request for the remote device to provide the previously-stored data via the communication network.

64. The system according to claim 63, wherein the remote device transmits the previously-stored data from a storage arrangement to the diagnostic device.

65. The system according to claim 64, wherein the remote device is a central processing arrangement, and wherein the storage arrangement is a database connected to the central processing device.

66. The system according to claim 64, wherein the storage arrangement is a storage device connected to the diagnostic device.

67. The system according to claim 63, wherein the diagnostic device obtains the previously-stored data from a storage arrangement which is directly connected to the diagnostic device.

68. The system according to claim 61,
wherein the processing arrangement is connected to an input device,
wherein, if the patient does not have previously-stored data associated therewith, the diagnostic device requests new data for the patient from the input device, and
wherein the diagnostic device receives at least one of manually-entered data or electronically-provided data for the patient from the input device.

69. The system according to claim 61, wherein the diagnostic device includes a storage arrangement which stores an executable code thereon, and a processing arrangement which retrieves the executable code for generating the particular information.

70. The system according to claim 69, wherein the processing arrangement of the diagnostic device runs the executable code to execute a loading dosage procedure for the sample.

71. The system according to claim 70, wherein the loading dosage procedure generates a recommended dosage of medication for the patient based on the previously-stored data and a result of the test.

72. The system according to claim 71, wherein the loading dosage procedure provides the recommended loading dosage of the medication for the patient to a plurality of remote devices.

73. The system according to claim 72, wherein the remote devices include at least two of a physician's processing device and a pharmacy's processing device.

74. The system according to claim 73, wherein a physician is capable of changing the recommended dosage of the medication for the patient using at least one of the physician's processing device and the diagnostic device.

75. The system according to claim 70, wherein the loading dosage procedure generates an international normalized ratio (“INR”) for the sample of the patient based on the previously-stored data and a result of the test to provide the particular information.

76. The system according to claim 75, wherein the diagnostic device transmits the generated INR for the patient to a remote device to be stored on a remote storage device as a portion of the previously-stored data of the patient.

77. The system according to claim 75, wherein at least one of the diagnostic device and a remote device records the generated INR for the patient on the storage arrangement as a portion of the previously-stored data of the patient.

78. The system according to claim 75,
wherein, if at least one device of the diagnostic device and a remote device determine that the generated INR of the patient is below a lowest value in a predetermined threshold range, the at least one of the diagnostic device and the remote device continues a periodic execution of the loading dosage procedure for the patient,
wherein, if at least one device of the diagnostic device and the remote device determine that the INR for the sample of the patient is above a highest value of the predetermined threshold range, an alert is generated to indicate a value of the INR, and
wherein, if at least one device of the diagnostic device and the remote device determine that the INR for the sample of the patient is in the predetermined threshold range, the recommended dosage of the medication corresponding to the INR for the sample of the patient is recorded to be utilized by a maintenance dosage procedure.

79. The system according to claim 78, wherein the maintenance dosage procedure generates a maintenance INR for the sample of the patient based on the previously-stored data and the INR generated by the loading dosage procedure, and wherein the maintenance INR corresponds to a maintenance dosage.

80. The system according to claim 79, wherein the maintenance dosage procedure generates a maintenance dosage of the medication for the patient based on the previously-stored data and the INR for the patient generated by the loading dosage procedure.

81. The system according to claim 79, wherein the at least one device provides the maintenance dosage of the medication for the patient to a plurality of remote devices.

82. The system according to claim 79, wherein the at least one device allows for a modification of the maintenance INR for the patient via an input device.

83. The system according to claim 79, wherein the at least one device provides the maintenance dosage of the medication for the patient to a plurality of remote devices.

84. The system according to claim 83, wherein the remote devices include at least two of a physician's processing device and a pharmacy's processing device.

85. The system according to claim 79, wherein the at least one device initiates the maintenance dosage procedure after the INR for the patient generated by the loading dosage procedure reaches a value that is within the predetermined threshold.

86. The system according to claim 79, wherein the at least one device transmits the maintenance INR for the patient to the remote device to be stored on a remote storage device to be a portion of the previously-stored data of the patient.

87. The system according to claim 79, wherein the at least one device records the maintenance INR for the patient on a storage arrangement connected to the remote device to be a portion of the previously-stored data of the patient.

88. The system according to claim 79, wherein if the at least one device determines that the maintenance INR for the patient is outside a predefined range, the at least one device modifies the maintenance dosage and re-executes the maintenance dosage procedure to determine if the maintenance INR corresponding to the modified maintenance dosage is within the predefined range.

89. The system according to claim 79, wherein the diagnostic device is connected to an input device, wherein a user is capable of modifying the maintenance INR for the patient via the input device.

90. The system according to claim 61, wherein the diagnostic device is connected to an insertion arrangement which is adapted to receive the biological property which includes a bodily fluid of the patient, and wherein, when the bodily fluid is received in the insertion arrangement, the diagnostic device provides data corresponding to a composition and amounts of certain elements of the biological property.

91. The system according to claim 61, wherein the diagnostic device is a biosensing portable device.

92. The system according to claim 91, wherein the portable device includes a personal digital assistant.

93. The process according to claim 61, wherein at least one of the diagnostic device and the remote device transmit the particular information to a web processing arrangement to be displayed on a web page associated with the patient, the web page being accessible by the patient via a secure connection.

94. A web arrangement for providing particular information associated with patients, comprising:
a processing system capable of retrieving previously-stored data associated with the patient, and the particular information provided by at least one of a diagnostic device and a remote device,
wherein the particular information is determined by executing a test performed on a biological property using at least one of the diagnostic device and the remote device, the particular information being determined based on the received previously-stored data, the particular information including at least one measurement of the biological property, and
wherein the processing system provides access to at least one of the respective patient of the patients associated with the particular information and a further user to view the particular information.

95. The web arrangement according to claim 94, wherein the processing system is capable of providing a personal web page for each of the patients so that the particular information for each respective patient is displayed thereto, and prevents access of the particular information to other patients.

96. The web arrangement according to claim 94, wherein the further user includes at least one of a physician of the respective patient, a pharmacy of the respective patient, a nurse of the respective patient and a relative of the respective patient.

97. A software system which, when executed on a processor, is capable of communicating determining particular information associated with a patient, the software comprising:
a program which is capable of configuring the processor to:
a) prompt the diagnostic device to receive previously-stored data associated with the patient, and
b) perform a test on a biological property to ascertain the particular information of the patient based on the received previously-stored data, the particular information including at least one measurement of the biological property.

98. The software system according to claim 97, wherein the particular information includes at least one of a medication dosage recommendation and a therapeutic recommendation for the patient.

99. A diagnostic device for ascertaining particular information related to a patient, comprising:
a processing arrangement which is capable of:
i. receiving previously-stored data associated with the patient from a remote device, and
ii. ordering a performance of a test on a biological sample to ascertain the particular information based on the received previously-stored data, the particular information including a level of at least one compound provided in the sample, the sample containing a bodily fluid of the patient.

100. A process for communicating between a diagnostic device and a remote device and determining particular information associated with a patient, comprising the steps of:
a) receiving previously-stored data associated with the patient by diagnostic device from the remote device; and
b) with the diagnostic device, performing a biological test on a biological sample to ascertain the particular information based on the received previously-stored data, the particular information including a level of at least one compound provided in the sample, the sample containing a bodily fluid of the patient.

101. A system for determining particular information associated with a patient, comprising:
a remote device providing previously-stored data associated with the patient; and
a diagnostic device communicating with the central processing arrangement and receiving the previously-stored data associated with the patient the diagnostic device from the remote device, the diagnostic device performing a test on a biological sample to ascertain the particular information based on the received previously-stored data, the particular information including a level of at least one compound provided in the sample, the sample containing a bodily fluid of the patient.

102. A web arrangement for providing particular information associated with patients, comprising:
a processing system capable of retrieving previously-stored data associated with the patient, and the particular information provided by at least one of a diagnostic device and a remote device,
wherein the particular information is determined using a test performed on a biological sample by at least one of the diagnostic device and the remote device based on the received previously-stored data, the particular information including a level of at least one compound provided in the sample, the sample containing a bodily fluid of the patient, and
wherein the processing system provides access to at least one of the respective patient of the patients associated with the particular information and a further user to view the provided particular information.

103. A software system which, when executed on a processor, is capable of communicating between a diagnostic device and a remote device and determining particular information associated with a patient, the software comprising:
a program which is capable of configure the processor to:
a) prompt the diagnostic device to receive previously-stored data associated with the patient, and
b) perform a test on a biological sample to ascertain the particular information based on the received previously-stored data, the particular information including a level of at least one compound provided in the sample, the sample containing a bodily fluid of the patient.
